(19) United States
(12) Patent Application Publication
(10) Pub. No.: US 2007/0292379 A1
(43) Pub. Date: Dec. 20, 2007

(54) HAIR TREATING AGENT COMPRISING CORNEOCYTE PROTEINS OR CORNEOCYTE POLYPEPTIDES

(76) Inventors: Petra Somfleth, Neu Wulmstorf (DE); Erik Schulze Zur Wiesche, Hamburg (DE); Elisabeth Poppe, Hamburg (DE)

Correspondence Address: PAUL & PAUL, 2000 MARKET STREET, PHILADELPHIA, PA, 19103-3229, US

(21) Appl. No.: 11/769,046

(22) Filed: Jun. 27, 2007

Related U.S. Application Data
(63) Continuation of application No. PCT/EP05/09793, filed on Sep. 13, 2005.

(30) Foreign Application Priority Data
Dec. 27, 2004 (DE) 10 2004 063 628.1

(51) Int. Cl. A61K 8/89; A61K 8/64

(52) U.S. Cl. 424/70.12; 424/70.14

(57) ABSTRACT

A hair conditioner that contains 0.05 to 5% by weight of at least one corneocyte protein or corneocyte polypeptide having a relative molar mass ranging between 10 and 40 kDa.

### CROSS-REFERENCE TO RELATED APPLICATIONS

This application is a continuation under 35 U.S.C. § 365 and 35 U.S.C. § 120 of International Application No. PCT/EP2005/009793, filed Sep. 13, 2005. This application also claims priority under 35 U.S.C. § 119 of German Application No. DE 10 2004 063628.1, filed Dec. 27, 2004.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to hair treating agents comprising corneocyte proteins, or proteins of comparable structures or corneocyte polypeptides as well as the use of these compositions for cleaning and/or caring for skin and hair.

(2) Description of Related Art, Including Information Disclosed Under 37 C.F.R. §§ 1.97 and 1.98.

Not Applicable

The cosmetic treatment of skin and hair is an important component of human body care. Nowadays, human hair is treated in a variety of ways with hair cosmetic preparations. They include, for example, the cleaning of hair with shampoos, care and regeneration with rinses and cures as well as bleaching, dyeing and styling the hair with colorants, toners, permanent wave lotions and styling preparations. Among these, agents for changing or nuancing the color of hair play a prominent role. From the viewpoint of the bleaching agents, which produce an oxidative lightening of the hair by degrading the natural hair colorants, there exist essentially three important types of hair dyes in the field of hair dyeing.

The so-called oxidation dyes are used for long-lasting, intensive colorations with corresponding authentic characteristics. Such dyes usually comprise oxidation dye precursors, “developer components” and “coupler components.” Under the influence of oxidizing agents or from atmospheric oxygen, the developer components form the actual colorants among each other or by coupling with one or more coupler components. The oxidation dyes are distinguished by outstanding, long-lasting coloration results. However, for colorations with a natural appearance, normally a mixture of a large number of oxidation dye precursors must be employed; in many cases, further substantive dyes are used for nuancing. If during the color development, the resulting or directly added dyes exhibit markedly different color fastness properties (e.g., UV stability, perspiration fastness, wash fastness etc.), then in time, a noticeable and therefore unwanted color shift can result. This phenomenon will be stronger when the hairstyle possesses hair or zones of hair with different degrees of damage. An example of this is long hair, in which the hair ends have suffered long term exposure to all possible environmental influences and are generally significantly more damaged than the relatively newly grown zones of hair.

For temporary colorations, usually colorants or toners are used that comprise “substantives” as the coloring component. These are dye molecules that are directly absorbed onto the hair and do not require any oxidative process to develop the color. These dyes include, for example, Henna that was already known in antiquity for dyeing skin and hair. In general, these dyes are significantly more sensitive to shampooing than are oxidative dyes, with the result that many unwanted nuance shifts occur very much faster or even a visible “decolorization” occurs.

Finally, another dyeing method has recently attracted lots of interest. In this method, precursors of the natural hair dye melanin are applied onto the hair; in the course of oxidative processes they then form analogs to natural colorants in the hair. In such methods, 5,6-dihydroxyindoline, for example, is employed as the dye precursor. By using, especially repeated use, of agents with 5,6-dihydroxyindoline it is possible to restore the natural hair color to people with gray hair. In this way the coloration can take place with atmospheric oxygen as the sole oxidizing agent, with the result that no recourse is needed to other oxidizing agents. The indoline can be employed as the sole dye precursor for people with naturally medium blond to brown hair. On the other hand, for use with people with naturally red and especially dark to black hair, satisfactory results can frequently only be obtained when additional dye components are used as well, in particular, specific oxidation dyestuff precursors.

The significance of care products with a long-lasting effect is growing due not least to the serious stressing of the hair by such color-changing treatments, permanent waving, but also to shampooing and harmful environmental factors. These care products influence the natural structure and properties of the hair. Thus, after treatment with a care product, the wet and dry combability of the hair, the hold and volume of the hair can be optimized or the hair can be protected against increased splitting.

Accordingly, it has long been common practice to subject the hair to a special after treatment, in which the hair is treated with special active components, for example, quaternary ammonium salts or special polymers, usually in the form of a rinse. Depending on the formulation, the combability, hold and volume of the hair are improved and the splitting rate reduced by this treatment.

So-called combination preparations have recently been developed to reduce the complexity of the usual multistage methods, particularly where the preparations are directly applied by the user.

Besides the usual components for cleaning the hair, for example, these preparations also contain additional active components that had previously been reserved for the hair after treatment preparations. Thus, the consumer saves an application step and at the same time, packaging costs are reduced because one less product is used.

The available active components, both for separate after treatment compositions and for combination preparations, generally act preferentially on the surface of the hair. Thus, active components are known, which provide the hair with shine, hold, volume, better wet or dry combability or prevention of splitting. Just as important as the outward appearance of the hair, however, is the inner structural cohesion of the hair fibers, which can be seriously affected, in particular, by oxidative and reductive processes, such as coloring and permanent waving.

However, the known active components cannot adequately meet all the requirements. Accordingly, there still remains a need for active substances or combinations of active substances for cosmetics with good caring properties and good biological degradability. There remains the need for further active care substances that can be incorporated without difficulty into known formulations, particularly in formulations containing dyes and/or electrolytes.

### BRIEF SUMMARY OF THE INVENTION

It has now been found that particularly advantageous results are produced when corneocyte proteins or corneocyte polypeptides of specific molecular weights are incorporated into hair treating agents.

A first subject matter of the present invention is hair treating agents comprising 0.05 to 5 wt. % of at least one corneocyte protein or corneocyte polypeptide with a molecular weight of 10 to 40 kDa.

According to the invention, the hair treating agents comprise at least one corneocyte protein or corneocyte polypeptide with a molecular weight of 10,000 to 40,000 Dalton. In the context of the present invention, the term “corneocyte protein or corneocyte polypeptide” is understood to mean a protein or a polypeptide that in addition to being present in the hair is also present in the stratum corneum.

The stratum corneum (SC), also called the horny layer, forms the outside layer of the epidermis and principally serves as the permeability barrier. This protective layer consists of 14 to 27 plies of solidly packed, plate like, non-nucleated, keratin rich and continuously desquamative corneocytes (keratinocytes). The thickness of the stratum corneum varies between 6 and 15 μm, wherein it is considerably thicker on the palms of the hand and soles of the feet than on other parts of the body.

Fundamentally, the composition of the SC can be described as a two-compartment model: Undifferentiated, protein-containing cells are embedded according to the Ziegelstein-Mörtel principle into a matrix that can also be considered as an interstitial lipid phase. The cells are responsible for the physical and chemical stability, whereas the intercellular, non-polar lipids as the filling substance prevent the penetration of water and materials dissolved in the water and consequently control the water retention and the water evaporation. The major constituents of the horny layer are insoluble keratinic fractions, a hydratable and swellable substance, water and lipids. Among these, mainly keratin and lipids are found in the intracellular space. Keratin makes up about 80% of the total mass of the cornecytes. The dense packing of the constituents in the cell leads to a high stability, strength and elasticity. The interior of the cell is additionally surrounded by a “cornified envelope” of loricrin, small, proline-rich proteins, filaggrin, elafin, involucrin and cystatin. Equimolar amounts of ceramides (sphingolipids), fatty acids and cholesterol are found in the intercellular space. Cholesterol esters, triglycerides, glycosphingolipids and cholesterol sulfate are present in small concentrations in the intercellular spaces. The presence of the lipids in the appropriate composition and their specific structural organization can be regarded as essential for the development of an intact barrier function (e.g., protection from loss of water).

### BRIEF DESCRIPTION OF THE SEVERAL VIEWS OF THE DRAWING(S)

Not Applicable

### DETAILED DESCRIPTION OF THE INVENTION

Preferred corneocyte proteins and corneocyte polypeptides are particularly loricrin and involucrin. Besides these, cornifin, trichohyalin, sciellin, profilaggrin and keratolinin are also preferred, such that inventively preferred hair treating agents are those wherein the corneocyte protein(s) or corneocyte polypeptide(s) is/are selected from the group involucrin, loricrin cornifin, trichohyalin, sciellin, profilaggrin and keratolinin.

According to the invention, corneocyte proteins or corneocyte polypeptides of a specific molecular weight range are employed. Proteins of the appropriate molecular weights can be obtained by known methods from the stratum corneum; polypeptides of the appropriate molecular weights can be obtained for example, by degrading higher molecular weight proteins or polypeptides.

Inventively preferred hair treating agents are those wherein the corneocyte protein or the corneocyte polypeptide has a molecular weight from 12.5 to 37.5 kDa, preferably 15 to 35 kDa and especially 20 to 30 kDa.

Corneocyte proteins or corneocyte polypeptides that according to the invention can be preferably employed, include—as already stated—involucrin, loricrin cornifin, trichohyalin, sciellin, profilaggrin and keratolinin and/or polypeptide fragments of these proteins. The proteins or polypeptides can be obtained from natural sources or be recombinantly produced. The proteins or polypeptides can be optionally branched, e.g., by a chemical branching agent or by an enzyme that forms bonds between neighboring polypeptides.

If desired, the proteins or polypeptides can be modified. Such modifications include, for example, chemical derivatization of one or a plurality of amino acids or modification of the amino acid sequence of the protein or polypeptide. The modifications can be used to lend the proteins or polypeptides specific properties, such as, for example, a higher water-solubility, a higher stability against the effects of chemicals, atmospheric or enzymatic stabilities, etc.

Depending on the molecular weight of the amino acids comprised in the corneocyte proteins or corneocyte polypeptides, these proteins or polypeptides can comprise several hundred to thousand amino acids. Preferred inventive hair treating agents are those wherein the corneocyte protein or corneocyte polypeptide includes 200 to 500 amino acids, preferably 250 to 450 amino acids, particularly preferably 275 to 400 amino acids and particularly 300 to 350 amino acids.

Particularly preferably, the corneocyte proteins or the corneocyte polypeptides comprised in the inventive hair treating agents comprise the amino acid glycine. A particularly preferred glycine content in the protein or polypeptide is from 1 to 40%, preferably from 1.5 to 35% and particularly from 2 to 30% glycine. In the context of the present invention, the statement “1% glycine” means that from 100 amino acids comprised in the protein or polypeptide molecule, one amino acid is glycine; analogously, the statement “30% glycine” in the context of the present invention means that from 100 amino acids comprised in the protein or polypeptide molecule, 30 amino acids are glycine.

The corneocyte proteins or the corneocyte polypeptides comprised in the inventive hair treating agent preferably also comprise the amino acid serine. A particularly preferred serine content in the protein or polypeptide is from 1 to 30%, preferably from 1.5 to 25% and particularly from 2 to 20% serine.

In further preferred inventive hair treating agents, the corneocyte proteins and similar proteins or corneocyte polypeptides possess the amino acid sequence “glycine-serine.” Proteins or polypeptides that are rich in this amino acid sequence are particularly preferred. Here, inventive hair treating agents are preferred, in which the corneocyte protein(s) or corneocyte polypeptide(s) possess the amino acid sequence glycine-serine, wherein preferred proteins are characterized in that the amino acids bonded in the amino acid sequence glycine-serine make up at least 5%, preferably at least 10% and particularly at least 15% of all amino acids comprised in the protein or polypeptide. In other words, the protein comprises the amino acid sequence “glycine-serine” at least five times (5%), preferably ten times (10%) and particularly 15 times (15%) per 200 amino acids.

The corneocyte proteins or the corneocyte polypeptides comprised in the inventive hair treating agent preferably also comprise the amino acid cysteine. A particularly preferred cysteine content in the protein or polypeptide is from 1 to 20%, preferably from 1.5 to 15% and particularly from 2 to 10% cysteine.

In further preferred inventive hair treating agents, the corneocyte protein or corneocyte polypeptide possesses the amino acid sequence “glycine-serine-cysteine.” Proteins or polypeptides that are rich in this amino acid sequence are particularly preferred. Here, inventive hair treating agents are preferred, in which the corneocyte protein(s) or corneocyte polypeptide(s) possess the amino acid sequence glycine-serine-cysteine, wherein preferred proteins are characterized in that the amino acids bonded in the amino acid sequence glycine-serine-cysteine make up at least 5%, preferably at least 10% and particularly at least 15% of all amino acids comprised in the protein or polypeptide. In other words, the protein comprises the amino acid sequence “glycine-serine-cysteine” at least five times (5%), preferably ten times (10%) and particularly 10 times (15%) per 300 amino acids.

The inventive compositions can comprise additional active substances and auxiliaries. These are described below.

The addition of surfactants (E) to the inventive compositions has proved to be particularly advantageous. Accordingly, in a further preferred embodiment, the inventive compositions comprise surfactants. The term “surfactant” is understood to mean surface-active substances that form adsorption layers at surfaces and interfaces or can aggregate in volume phases to micelle colloids or lyotropic mesophases. One differentiates between anionic surfactants that consist of a hydrophobic group and a negatively charged hydrophilic head group, amphoteric surfactants that carry both a negative and a compensating positive charge, cationic surfactants that besides a hydrophobic group have a positively charged hydrophilic group, and non-ionic surfactants that have no charges but rather strong dipole moments and are strongly hydrated in aqueous solution.

All anionic surface-active materials that are suitable for use on the human body are suitable anionic surfactants (E1) for the inventive preparations. They are characterized by a water solubilizing anionic group, such as e.g., a carboxylate, sulfate, sulfonate or phosphate group and a lipophilic alkyl group containing about 8 to 30 carbon atoms. In addition, the molecule may contain glycol or polyglycol ether groups, ester, ether and amide groups as well as hydroxyl groups. Exemplary suitable anionic surfactants are, each in the form of the sodium, potassium and ammonium as well as the mono, di and trialkanolammonium salts with 2 to 4 C atoms in the alkanol group,
linear and branched fatty acids with 8 to 30 C atoms (soaps),
ether carboxylic acids of the formula R—O—(CH₂—CH₂O)ₓ—CH₂—COOH, in which R is a linear alkyl group with 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
sulfosuccinic acid mono- and dialkyl esters with 8 to 24 carbon atoms in the alkyl group and sulfosuccinic acid mono-alkyl polyoxyethyl esters with 8 to 24 carbon atoms in the alkyl group and 1 to 6 oxyethylene groups,
linear alkane sulfonates with 8 to 24 carbon atoms,
linear alpha-olefin sulfonates with 8 to 24 carbon atoms
alpha-sulfo fatty acid methyl esters of fatty acids with 8 to 30 carbon atoms,
alkyl sulfates and alkyl polyglycol ether sulfates of formula R—O(CH₂—CH₂O)ₓ—OSO₃H, in which R is preferably a linear alkyl group with 8 to 30 carbon atoms and x =0 or 1 to 12,
sulfated hydroxyalkyl polyethylene- and/or hydroxyalkylene propylene glycol ethers
sulfonated unsaturated fatty acids with 8 to 24 carbon atoms and 1 to 6 double bonds
esters of tartaric acid and citric acid with alcohols, which represent the addition products of about 2-15 molecules of ethylene oxide and/or propylene oxide on fatty alcohols with 8 to 22 C atoms,
alkyl- and/or alkenyl ether phosphates of formula (E1-I),
   R¹(OCH₂CH₂)ₙ—O—P(O)(OX)—OR² (E1-I)
   in which R¹ preferably stands for an aliphatic hydrocarbon group with 8 to 30 carbon atoms, R² stands for hydrogen, a (CH₂CH₂O)ₙR² group or X, n for numbers between 1 and 10 and X for hydrogen, an alkali- or alkaline earth metal or NR³R⁴R⁵R⁶, with R³ to R⁶, independently of each other standing for a C₁ to C₄ hydrocarbon group, sulfated fatty acid alkylene glycol esters of formula (E1-II)
   R⁷CO(AlkO)ₙSO₃M (E1-II)
   in which R⁷CO stands for a linear or branched, aliphatic, saturated and/or unsaturated acyl group with 6 to 22 carbon atoms, Alk for CH₂CH₂, CHCH₃CH₂ and/or CH₂CHCH₃, n for numbers from 0.5 to 5 and M for a cation,
monoglyceride sulfates and monoglyceride ether sulfates of formula (E1-III) 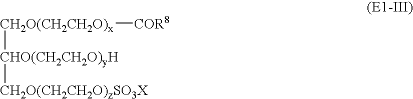
   in which R⁸CO stands for a linear or branched acyl group with 6 to 22 carbon atoms, the sum of x, y and z is 0 or stands for numbers between 1 and 30, preferably 2 to 10, and X stands for an alkali- or alkaline earth metal. In the context of the invention, typical examples of suitable monoglyceride (ether) sulfates are the reaction products of lauric acid monoglyceride, cocoa fatty acid monoglyceride, palmitic acid monoglyceride, stearic acid monoglyceride, oleic acid monoglyceride and tallow fatty acid monoglyceride as well as their ethylene oxide adducts with sulfur trioxide or chlorosulfonic acid in the form of their sodium salts. Preferably, monoglyceride sulfates of formula (E1-III) are added, in which R⁸CO stands for a linear acyl group with 8 to 18 carbon atoms,
amido ether carboxylic acids,
Condensation products of C₈-C₃₀ fatty alcohols with protein hydrolyzates and/or amino acids and their derivatives, which are known to the person skilled in the art as albumin fatty acid condensates, such as for example, the Lamepon® types, Gluadin® types, Hostapon® KCG or the Amisoft® types.

Preferred anionic surfactants are alkyl sulfates, alkyl polyglycol ether sulfates and ether carboxylic acids with 10 to 18 C atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerine disulfates, alkyl- and alkenyl ether phosphates as well as albumin fatty acid condensates.

Those surface-active compounds that carry at least one quaternary ammonium group and at least one —COO⁽⁻⁾ or —SO₃⁽⁻⁾ group in the molecule are designated as zwitterionic surfactants (E2). Particularly suitable zwitterionic surfactants are the so-called betaines such as the N-alkyl-N,N-dimethyl ammonium glycinates, for example, the cocoalkyl dimethyl ammonium glycinate, N-acylaminopropyl-N,N-dimethyl ammonium glycinates, for example, the cocoacylaminopropyl dimethyl ammonium glycinate, and 2-alkyl-3-carboxymethyl-3-hydroxyethyl imidazolines with 8 to 18 carbon atoms in each of the alkyl or acyl groups, as well as cocoacylaminoethyl hydroxyethyl carboxymethyl glycinate. A preferred zwitterionic surfactant is the fatty acid amide derivative, known under the INCI name cocoamidopropyl betaine.

The ampholytic surfactants (E3) are understood to include such surface-active compounds that apart from a C₈₋₁₈ alkyl or acyl group, comprise at least one free amino group and at least one —COOH or —SO₃H group in the molecule, and are able to form internal salts. Examples of suitable ampholytic surfactants are N-alkylglycines, N-alkylamino propionic acids, N-alkylamino butyric acids, N-alkylimino dipropionic acids, N-hydroxyethyl-N-alkylamidopropylglycine, N-alkyltaurines, N-alkylsarcosines, 2-alkylamino propionic acids and alkylamino acetic acids, each with about 8 to 24 carbon atoms in the alkyl group. Particularly preferred ampholytic surfactants are N-cocoalkylamino propionate, cocoacylaminoethylamino propionate and C₁₂-C₁₈ acyl sarcosine.

Non-ionic surfactants (E4) comprise e.g., a polyol group, a polyalkylene glycol ether group or a combination of polyol and polyglycol ether groups as the hydrophilic group. Exemplary compounds of this type are:
addition products of 2 to 50 moles ethylene oxide and/or 0 to 5 moles propylene oxide to linear and branched fatty alcohols containing 8 to 30 carbon atoms, to fatty acids with 8 to 30 carbon atoms and to alkyl phenols with 8 to 15 carbon atoms in the alkyl group,
methyl or C₂-C₆ alkyl group end blocked addition products of 2 to 50 moles ethylene oxide and/or 0 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as, for example, the commercially available types Dehydol® LS, Dehydol® LT (Cognis),
C₁₂-C₃₀ fatty acid mono and diesters of addition products of 1 to 30 moles ethylene oxide on glycerine;
Addition products of 5 to 60 moles ethylene oxide on castor oil and hydrogenated castor oil,
polyol esters of fatty acids, such as, for example, the commercial product Hydagen® HSP (Cognis) or Sovermol types (Cognis),
alkoxylated triglycerides,
alkoxylated fatty acid alkyl esters of formula (E4-I)
   R¹CO—(OCH₂CHR²)_{w}OR³ (E4-I)
   in which R¹CO stands for a linear or branched, saturated and/or unsaturated acyl group with 6 to 22 carbon atoms, R² for hydrogen or methyl, R³ for linear or branched alkyl groups with 1 to 4 carbon atoms and x for numbers from 1 to 20,
amine oxides,
hydroxy mixed ethers,
sorbitol esters of fatty acids and addition products of ethylene oxide to sorbitol esters of fatty acids such as e.g., the polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to formula (E4-II),
   R⁴O-[G]ₚ (E4-II)
   in which R⁴ stands for an alkyl or alkenyl group with 4 to 22 carbon atoms, G for a sugar group with 5 or 6 carbon atoms and p for numbers from 1 to 10. They can be obtained according to the appropriate methods of preparative organic chemistry.

The alkyl and alkenyl oligoglycosides can be derived from aldoses or ketoses containing 5 or 6 carbon atoms, preferably from glucose. The preferred alkyl and/or alkenyl oligoglycosides are accordingly alkyl and/or alkenyl oligoglucosides The index value p in the general formula (E4-II) represents the degree of oligomerization (DP), i.e., the distribution of mono and oligoglycosides, and stands for a number between 1 and 10. Whereas in a single molecule, p must always be a whole number and here above all can assume the values p=1 to 6, the value p for a specific alkyl oligoglycoside is an analytically determined, calculated quantity that mostly represents a fractional number. Preferably, alkyl and/or alkenyl oligoglycosides are employed with an average degree of oligomerization p from 1.1 to 3.0. From the industrial point of view, such alkyl and/or alkenyl oligoglycosides are preferred with degrees of oligomerization less than 1.7 and in particular, between 1.2 and 1.4. The alkyl or alkenyl group R⁴ can be derived from primary alcohols containing 4 to 11, preferably 8 to 10 carbon atoms. Typical examples are butanols, caproyl alcohol, caprylic alcohol, capric alcohol and undecyl alcohol as well as their industrial mixtures, such as for example, those obtained by the hydrogenation of industrial fatty acid methyl esters or in the course of the hydrogenation of aldehydes from the Roelen Oxo-synthesis. Alkyl oligoglucosides with chain lengths C₈-C₁₀ (DP=1 to 3) are preferred, which result as the low boiling fraction in the separative distillation of industrial C₈-C₁₈ coco fatty alcohol and which can be contaminated with a fraction of less than 6 wt. % of C₁₂ alcohol as well as alkyl oligoglucosides based on C_{9/11} oxoalcohols (DP=1 to 3). The alkyl or alkenyl group R¹⁵ can moreover be derived from primary alcohols containing 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol as well as their industrial mixtures that can be obtained as described above. Alkyl oligoglucosides based on hydrogenated C_{12/14}-coco alcohol with a DP of 1 to 3 are preferred.
sugar surfactants of the type fatty acid N-alkylpolyhydroxyalkyl amides, a non-ionic surfactant of formula (E4-III),
   R⁵CO—NR⁶—[Z] (E4-III)
   in which R⁵CO stands for an aliphatic acyl group with to 6 to 22 carbon atoms, R⁶ for hydrogen, an alkyl or hydroxyalkyl group with to 1 to 4 carbon atoms and [Z] for a linear or branched polyhydroxyalkyl group with 3 to 12 carbon atoms and 3 to 10 hydroxyl groups. The fatty acid N-alkylpolyhydroxyalkyl amides are known substances, which may normally be obtained by reductive amination of a reducing sugar with ammonia, an alkylamine or an alkanolamine and subsequent acylation with a fatty acid, a fatty acid alkyl ester or a fatty acid chloride. The fatty acid N-alkylpolyhydroxyalkyl amides are advantageously derived from reducing sugars having 5 or 6 carbon atoms, especially from the glucoses. Accordingly, the fatty acid N-alkyl glucamides illustrate the fatty acid N-alkylpolyhydroxyalkyl amides, as are shown by formula (E4-IV):
   R⁷CO—NR⁸—CH₂—(CHOH)₄—CH₂OH (E4-IV)
   Preferably, glucamides of formula (E4-IV) are employed as the fatty acid N-alkylpolyhydroxyalkyl amides, in which R⁸ stands for hydrogen or an alkyl group and R⁷CO stands for the acyl group of caproic acid, caprylic acid, capric acid, lauric acid, myristic acid, palmitic acid, palmoleic acid, stearic acid, isostearic acid, oleic acid, elaidic acid, petroselic acid, linoleic acid, linolenic acid, arachic acid, gadoleic acid, behenic acid or erucic acid or their industrial mixtures. Fatty acid N-alkylglucamides of formula (E4-IV) are particularly preferred, which are obtained by reductive amination of glucose with methylamine and subsequent acylation with lauric acid or C12/14 coco fatty acid or a corresponding derivative. In addition, the polyhydroxyalkyl amides can also derive from maltose and palatinose.

Alkylene oxide addition products to saturated, linear fatty alcohols and fatty acids, each with 2 to 30 moles ethylene oxide per mole fatty alcohol or fatty acid, have proved to be preferred non-ionic surfactants. Preparations with excellent properties are also obtained when they comprise fatty acid esters of ethoxylated glycerine as the non-ionic surfactant.

These compounds are characterized by the following parameters. The alkyl group R comprises 6 to 22 carbon atoms and may be both linear and also branched. Primary linear aliphatic groups and aliphatic groups that are methyl-branched in the 2-position, are preferred. Such alkyl groups are for example, 1-octyl, 1-decyl, 1-lauryl, 1-myristyl, 1-cetyl and 1-stearyl. 1-Octyl, 1-decyl, 1-lauryl, 1-myristyl are particularly preferred. On using so-called “oxo alcohols” as starting materials, compounds with an odd number of carbon atoms in the alkyl chain preponderate.

Furthermore, quite particularly preferred non-ionic surfactants are the sugar surfactants. The compositions used according to the invention preferably comprise the sugar surfactants in quantities of 0.1 to 20 wt. %, based on the total composition. Quantities of 0.5 to 15 wt. % are preferred and quantities of 0.5 to 7.5 wt. % are quite particularly preferred.

For compounds with alkyl groups used as surfactants, they may each be pure substances. However, it is normally preferred to start with natural vegetal or animal raw materials for the manufacture of these materials, with the result that mixtures of substances are obtained, which have different alkyl chain lengths that depend on each raw material.

For surfactants, which are represented by the addition products of ethylene oxide and/or propylene oxide to fatty alcohols or derivatives of these addition products, both products with a “normal” homolog distribution as well as those with a narrow homolog distribution may be used. The term “normal” homolog distribution is understood to mean mixtures of homologs obtained from the reaction of fatty alcohols and alkylene oxide using alkali metals, alkali metal hydroxides or alkali metal alcoholates as catalysts. On the other hand, narrow homolog distributions are obtained if e.g., hydrotalcite, alkaline earth metal salts of ether carboxylic acids, alkaline earth metal oxides, hydroxides or alcoholates are used as catalysts. The use of products with a narrow homolog distribution can be preferred.

According to the invention, cationic surfactants of the type quaternary ammonium compounds, the esterquats and the amido amines can be employed. Preferred quaternary ammonium compounds are ammonium halides, particularly chlorides and bromides, such as alkyl trimethyl ammonium chlorides, dialkyl dimethyl ammonium chlorides and trialkyl methyl ammonium chloride, e.g., cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride, as well as the imidazolium compounds known under the INCI designations Quaternium-27 and Quaternium-83. The long alkyl chains of the abovementioned surfactants have preferably 10 to 18 carbon atoms.

According to the invention, QACs with behenyl groups are preferably employed, especially those substances known as behentrimonium chloride or bromide (docosanyl trimethyl ammonium chloride or bromide). Other preferred QACs possess at least two behenyl groups, wherein QACs with two behenyl groups on an imidazolinium backbone are particularly preferred. These substances are commercially available, for example, under the names Genamin® KDMP (Clariant) and Crodazosoft® DBQ (Crodauza).

Esterquats are known compounds, which both comprise at least one ester function and also a quaternary ammonium group as structural elements. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed, for example, under the trade names Stepantex®, Dehyquart® and Armocare®. The products Armocare® VGH-70, an N, N-bis(2-palmitoyloxyethyl) dimethyl ammonium chloride, as well as Dehyquart® F-75, Dehyquart® C-4046, Dehyquart® L80 and Dehyquart® AU 35 are examples of such esterquats.

The alkylamido amines are normally manufactured by the amidation of natural or synthetic fatty acids and fatty acid fractions with dialkylaminoamines. According to the invention, a particularly suitable compound from this substance group is represented by stearamidopropyldimethylamine, commercially available under the designation Tegamid® S 18.

The inventive compositions preferably comprise the cationic surfactants in quantities of 0.05 to 10 wt. %, based on the total composition. Quantities of 0.1 to 5 wt. % are particularly preferred. In the context of the present invention, “based on the total composition” means based on the mixture of the preparation of oxidation dye precursors (A) and the oxidizing agent preparation (B).

The surfactants (E) are used in quantities of 0.1 to 45 wt. %, preferably 0.5 to 30 wt. % and quite particularly preferably from 0.5-25 wt. %, based on the total inventively used composition.

Anionic, non-ionic, zwitterionic and/or amphoteric surfactants as well as mixtures thereof can be inventively preferred.

In summary, inventive hair treating agents are preferred that comprise, based on their weight, 0.5 to 70 wt. %, preferably 1 to 60 wt. % and particularly 5 to 25 wt. % of anionic and/or non-ionic and/or cationic and/or amphoteric surfactant(s).

Vitamins, provitamins or vitamin precursors are a further preferred group of ingredients of the inventive hair treating agents. These are described below.

In the group of substances designated as vitamin A, belong retinol (vitamin A₁) as well as 3,4-didehydroretinol (vitamin A₂). β-carotene is the provitamin of retinol. Examples of suitable vitamin A components according to the invention are vitamin A acid and its esters, vitamin A aldehyde and vitamin A alcohol as well as its esters such as the palmitate and acetate. The compositions according to the invention preferably comprise the vitamin A components in amounts of 0.05 to 1 wt. %, based on the total preparation.

The vitamin B group or the vitamin B complex include among other things
Vitamin B₁ (Thiamin)
Vitamin B₂ (Riboflavin)
Vitamin B₃. The compounds nicotinic acid and nicotinamide (niacinamide) are often included under this designation. According to the invention, nicotinamide is preferred and is comprised in the inventively used compositions in amounts of 0.05 to 1 wt. % based on the total composition.
Vitamin B₅ (pantothenic acid, panthenol and pantolactone). In the context of this group, panthenol and/or pantolactone is preferably used. Useable derivatives of panthenol according to the invention are especially the esters and ethers of panthenol as well as cationic derivatized panthenols. Specific representatives are for example, panthenol triacetate, panthenol monoethyl ether and its monoacetate as well as the cationic panthenol derivatives disclosed in WO 92/13829. The cited compounds of the vitamin B₅ type are preferably comprised in the compositions according to the invention in amounts of 0.05 to 10 wt. %, based on the total composition. Quantities of 0.1 to 5 wt. % are particularly preferred.
Vitamin B₆ (pyridoxine as well as pyridoxamine and pyridoxal).
Vitamin C (ascorbic acid). Vitamin C is preferably added to the compositions according to the invention in amounts of 0.1 to 3 wt. %, based on the total composition. Its use in the form of the palmitate ester the glucosides or phosphates can be preferred. Its use in combination with tocopherols can also be preferred.
Vitamin E (Tocopherols, especially α-tocopherol). Tocopherol and its derivatives, among which particularly the esters such as the acetate, the nicotinate, the phosphate and the succinate, are used in the compositions according to the invention preferably comprised in amounts of 0.05-1 wt. %, based on the total composition.
Vitamin F. The term “vitamin F” is usually taken to mean essential fatty acids, particularly linoleic acid, linolenic acid and arachidonoic acid.
Vitamin H. The compound (3aS,4S,6aR)-2-oxohexahydrothienol[3,4-d]imidazole-4-valeric acid denotes Vitamin H, for which the trivial name biotin has become accepted. The compositions according to the invention preferably comprise biotin in amounts of 0.0001 to 1.0 wt. %, particularly in amounts of 0.001 to 0.01 wt. %.

In summary, preferred hair treating agents according to the invention comprise the vitamins, provitamins and vitamin precursors classified in the groups A, B, C, E, F and H, wherein preferred compositions comprise the cited compounds in quantities of 0.1 to 5 wt. %, preferably from 0.25 to 4 wt. % and particularly from 0.5 to 2.5 wt. %, each based on the total composition.

In addition, particularly preferred inventive hair treating agents comprise silicone(s). Particularly preferred inventive hair treating agents further comprise silicone(s), preferably in amounts of 0.1 to 10 wt. %, preferably 0.25 to 7 wt. % and particularly 0.5 to 5 wt. %, each based on the total composition.

Inventive hair compositions are especially preferred that comprise a silicone, selected from:
(i) volatile or non-volatile, linear or cyclic, crosslinked or non-crosslinked polyalkylsiloxanes, polyarylsiloxanes, polyalkylarylsiloxanes,
(ii) polysiloxanes, which comprise one or more organofunctional groups in their general structure, selected from:
a) substituted or unsubstituted aminated groups;
b) (per)fluorinated groups;
c) thiol groups;
d) carboxylate groups;
e) hydroxylated groups;
f) alkoxylated groups;
g) acyloxyalkyl groups;
h) amphoteric groups;
i) bisulfite groups;
j) hydroxyacylamino groups;
k) carboxy groups;
l) sulfonic acid groups; and
m) sulfate or thiosulfate groups;
(ii) linear polysiloxane(A)-polyoxyalkylene(B) block copolymers of the type (A-B)ₙ with n>3;
(iii) grafted silicon polymers with non silicon-containing organic structures that consist of an organic backbone that is formed from organic monomers that do not comprise silicon, on which in the chain as well as optionally on at least one chain end at least one polysiloxane macromer has been grafted;
(iv) grafted silicon polymers with polysiloxane backbone, grafted onto the non silicon-containing organic monomer, which possess a polysiloxane main chain on which in the chain as well as optionally on at least one chain end at least one organic macromer has been grafted that comprises no silicon;
(v) or their mixtures.

Particularly preferred inventive hair treating agents are thus characterized in that they comprise at least one silicone of formula (I)
(CH₃)₃Si—[O—Si(CH₃)₂]ₓ—O—Si(CH₃)₃ (I),
in which x stands for a number from 0 to 100, advantageously from 0 to 50, more preferably from 0 to 20 and especially 0 to 10.

The inventively preferred hair treating agents comprise a silicone of the abovementioned formula 1. These silicones are designated according to the INCI nomenclature as DIMETHICONE. In the context of the present invention, preferred compounds employed as the silicone of formula I are:
(CH₃)₃Si—O—Si(CH₃)₃
(CH₃)₃Si—O—(CH₃)₂Si—O—Si(CH₃)₃
(CH₃)₃Si—[O—(CH₃)₂Si]₂—O—Si(CH₃)₃
(CH₃)₃Si—[O—(CH₃)₂Si]₃—O—Si(CH₃)₃
(CH₃)₃Si—[O—(CH₃)₂Si]₄—O—Si(CH₃)₃
(CH₃)₃Si—[O—(CH₃)₂Si]₅—O—Si(CH₃)₃
(CH₃)₃Si—[O—(CH₃)₂Si]₆—O—Si(CH₃)₃
(CH₃)₃Si—[O—(CH₃)₂Si]₇—O—Si(CH₃)₃
(CH₃)₃Si—[O—(CH₃)₂Si]₈—O—Si(CH₃)₃
(CH₃)₃Si—[O—(CH₃)₂Si]₉—O—Si(CH₃)₃
(CH₃)₃Si—[O—(CH₃)₂Si]₁₀—O—Si(CH₃)₃
(CH₃)₃Si—[O—(CH₃)₂Si]₁₁—O—Si(CH₃)₃
(CH₃)₃Si—[O—(CH₃)₂Si]₁₂—O—Si(CH₃)₃
(CH₃)₃Si—[O—(CH₃)₂Si]₁₃—O—Si(CH₃)₃
(CH₃)₃Si—[O—(CH₃)₂Si]₁₄—O—Si(CH₃)₃
(CH₃)₃Si—[O—(CH₃)₂Si]₁₅—O—Si(CH₃)₃
(CH₃)₃Si—[O—(CH₃)₂Si]₁₆—O—Si(CH₃)₃
(CH₃)₃Si—[O—(CH₃)₂Si]₁₇—O—Si(CH₃)₃
(CH₃)₃Si—[O—(CH₃)₂Si]₁₈—O—Si(CH₃)₃
(CH₃)₃Si—[O—(CH₃)₂Si]₁₉—O—Si(CH₃)₃
(CH₃)₃Si—[O—(CH₃)₂Si]₂₀—O—Si(CH₃)₃
   wherein (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ are particularly preferred.

Naturally, mixtures of the above cited silicones can also be comprised in the inventive compositions.

Preferred inventively employable silicones have viscosities at 20° C. of 0.2 to 2 mm²s⁻¹, wherein silicones with viscosities of 0.5 to 1 mm²s⁻¹ are particularly preferred.

Particularly preferred agents according to the invention comprise one or more aminofunctional silicones. Such silicones can be described by the formula
M(RₐQ_{b}SiO_{(4-a-b)/2)x}(R_{c}SiO_{(4-c)/2)y}M
wherein, in the above formula R is a hydrocarbon or a hydrocarbon group with 1 to 6 carbon atoms, Q is a polar group of the general formula —R¹HZ, wherein R¹ is a divalent, linking group that is bonded to hydrogen and the group Z, made up of carbon atoms and hydrogen atoms, carbon-, hydrogen- and oxygen atoms or carbon-, hydrogen- and nitrogen atoms, and Z is an organic amino functionalized group that comprises at least one amino functional group; “a” assumes values in the range of about 0 to about 2, “b” assumes values in the range of about 1 to about 3, “a”+“b” is less than or equal to 3, and “c” is a number in the range of about 1 to about 3, and x is a number in the range of 1 to about 2,000, advantageously from about 3 to about 50 and most preferably from about 3 to about 25, and y is a number in the range of about 20 to about 10,000, advantageously from about 125 to about 10,000 and most preferably from about 150 to about 1,000, and M is a suitable silicone end-group, as is known from the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl and the like; alkenyl groups, such as vinyl, halogenovinyl, alkylvinyl, allyl, halogenoallyl, alkylallyl; cycloalkyl groups, such as cyclobutyl, cyclopentyl, cyclohexyl and the like; phenyl groups, benzyl groups, halogenated hydrocarbon groups, such as 3- chloropropyl, 4-bromobutyl, 3,3,3-trifluoropropyl, chlorocyclohexyl, bromophenyl, chlorophenyl and the like as well as sulfur-containing groups, such as mercaptoethyl, mercaptopropyl, mercaptohexyl, mercaptophenyl and the like; advantageously R is an alkyl group that comprises 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—.

Z is an organic, aminofunctional group comprising at least one functional amino group. A possible formula for Z is NH(CH₂)_{z}NH₂, wherein z is 1 or more. Another possible formula for Z is —NH(CH₂)_{z}(CH₂)_{zz}NH, wherein both z and zz independently are 1 or more, wherein this structure includes diamino ring structures, such as piperazinyl. Most preferably, Z is an —NHCH₂CH₂NH₂ group. Another possible formula for Z is —N(CH₂)_{z}(CH₂)_{zz}NX₂ or —NX₂, in which each X of X₂ is independently selected from the group consisting of hydrogen and alkyl groups with 1 to 12 carbon atoms, and zz is 0.

Most preferably, Q is a polar, amine functional group of formula —CH₂CH₂CH₂NHCH₂CH₂NH₂. In the formulas “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b} SiO_{(4-a-b)/2} units to the R_{c}SiO_{(4-c)/2} units is in the range from about 1:2 to 1:65, preferably from about 1:5 to about 1:65 and most preferably from about 1:15 to about 1:20.lf one or a plurality of silicones of the above formula are added, then the different variable substituents in the above formula for the different silicone components that are present in the silicone mixture can be different.

Preferred inventive hair treating agents are characterized in that they comprise an aminofunctional silicone of formula (II)
R′ₐG₃₋ₐ—Si(OSiG₂)ₙ—(OSiG_{b}R′_{2-b})ₘ—O—SiG₃₋ₐ—R′ₐ (II),
wherein:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, particularly 0;
b stands for a number between 0 and 1, particularly 1;
m and n are numbers whose sum (m +n) is between 1 and 2,000, preferably between 50 and 150, wherein n preferably assumes values of 0 to 1999 and particularly 49 to 149 and m preferably assumes values of 1 to 2,000, particularly 1 to 10,
R′ is a monovalent group selected from
-Q-N(R″)—CH₂—CH₂—N(R″)₂
-Q-N(R″)₂
-Q-N⁺(R″)₃A⁻
-Q-N⁺H(R″)₂A⁻
-Q-N⁺H₂(R″)A⁻
-Q-N(R″)—CH₂—CH₂—N⁺R″H₂A⁻,
   wherein each Q stands for a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,

R″ stands for the same or different groups from the group -H, -phenyl, -benzyl, —CH₂—CH(CH₃)Ph, the C₁₋₂₀-alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion that is preferably selected from chloride, bromide, iodide or methosulfate.

Particularly preferred inventive hair treating agents are characterized in that they comprise at least one aminofunctional silicone of formula (IIa) 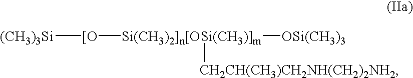
in which m and n are numbers whose sum (m+n) is between 1 and 2,000, preferably between 50 and 150, wherein n preferably assumes values of 0 to 1,999 and particularly 49 to 149 and m preferably assumes values of 1 to 2,000, particularly 1 to 10.

These silicones are designated according to the INCI nomenclature as Trimethylsilylamodimethicones.

Particularly preferred inventive hair treating agents are also those that comprise at least one amino functional silicone of formula (IIb) 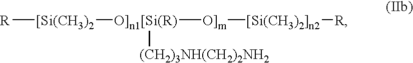
in which R stands for —OH, —O—CH₃ or a —CH₃ group and m, n1 und n2 are numbers whose sum (m+n1+n2) is between 1 to 2,000, preferably between 50 and 150, wherein the sum (n1+n2) preferably assumes values of 0 to 1,999 and particularly 49 to 149 and m preferably assumes values of 1 to 2,000, particularly 1 to 10.

These silicones are designated according to the INCI nomenclature as Amodimethicones.

Independently of which aminofunctional silicone is added, inventive hair treating agents are preferred that comprise an aminofunctional silicone whose amino number is above 0.25 meq/g, preferably above 0.3 meq/g and particularly above 0.4 meq/g. The amine number stands for the milliequivalents of amine per gram of aminofunctional silicone. It can be measured by titration and is also reported with the unit mg KOH/g.

According to the invention, preferred hair treating agents are characterized in that they comprise, based on their weight, 0.01 to 10 wt. %, preferably 0.1 to 8 wt. %, particularly preferably 0.25 to 7.5 wt. % and particularly 0.5 to 5 wt. % aminofunctional silicone(s).

Also, according to the invention, the addition of cyclic dimethicones, designated by INCI as CYCLOMETHICONES, is preferred. Here, inventive hair treatment agents are preferred that comprise at least one silicone of formula III 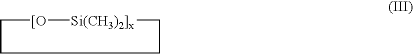
in which x stands for a number from 0 to 200, advantageously from 0 to 10, more preferably from 0 to 7 and especially 0, 1, 2, 3, 4, 5 or 6.

The above described silicones possess a backbone that is constructed from —Si—O—Si— units. Of course, these Si—O—Si units can also be interrupted by carbon chains. Appropriate molecules are obtained by chain extension reactions and are preferably added in the form of silicone in water emulsions.

The silicone in water emulsions that are added according to the invention can be prepared by means of known processes, as disclosed, for example, in U.S. Pat. No. 5,998,537 and EP 0 874 017 A1.

In summary, this manufacturing process includes the emulsifiable mixture of components, one comprising at least one polysiloxane, the other comprising at least one organosilicon material that reacts with the polysiloxane in a chain extension reaction, wherein at least one chain extension reaction catalyst that contains a metal ion is present, as well as a surfactant and water.

Chain extension reactions with polysiloxanes are known and can include, for example, the hydrosilylation reaction, in which a Si—H group is reacted with an aliphatic unsaturated group in the presence of a platinum/rhodium catalyst to form polysiloxanes with several Si—(C)ₚ—Si bonds (p=1-6), the polysiloxanes being also designated as polysiloxane-polysilalkylene copolymers.

The chain extension reaction can also include the reaction of an Si—OH group (for example, a hydroxyl terminated polysiloxane) with an alkoxy group (for example, alkoxy silanes, silicates or alkoxysiloxanes) in the presence of a metal-containing catalyst to afford polysiloxanes.

The polysiloxanes that are used in the chain extension reaction include a substantially linear polymer of the following structure:
R—Si(R₂)—[—O—Si(R₂)—]ₙ—O—SiR₃

In this structure, each R independently of each other stands for a hydrocarbon group with up to 20 carbon atoms, preferably with 1 to 6 carbon atoms, such as, for example, an alkyl group (for example, methyl, ethyl, propyl or butyl), an aryl group (for example, phenyl), or the group required for the chain extension reaction (“reactive group,” for example, Si-bonded hydrogen atoms, aliphatic unsaturated groups like vinyl, allyl or hexenyl, hydroxy, alkoxy like methoxy, ethoxy or propoxy, alkoxy-alkoxy, acetoxy, amino etc.) with the proviso that on average, one or two reactive groups per polymer are present, n is a positive number >1. Preferably, an excess of reactive groups, particularly preferably >90%, and in particular, >98% of the reactive groups, is bonded to the terminal Si atom in the siloxane. Preferably, n stands for numbers that describe what viscosities between 1 and 1,000,000 mm²/s the polysiloxanes have, particularly preferably viscosities between 1,000 and 100,000 mm²/s.

The polysiloxanes can be branched to a small extent (for example, <2 mol % of the siloxane units), or the polymers are substantially linear, particularly preferably completely linear. In addition, the substituents R can themselves be substituted, for example, by N-containing groups (for example, amino groups), epoxy groups, S-containing groups, Si-containing groups, O-containing groups etc. Preferably, at least 80% of the R groups are alkyl groups, particularly preferably methyl groups.

The organosilicon material that reacts with the polysiloxane in the chain extension reaction can either be a second polysiloxane, or a molecule that acts as a chain extender. If the organosilicon material is a polysiloxane, then it has the abovementioned general structure. In these cases one polysiloxane possesses (at least) one reactive group in the reaction, and a second polysiloxane possesses (at least) one second reactive group that reacts with the first.

When the organosilicon material includes a chain extender, then this can be a material such as, for example, a silane, a siloxane (for example, disiloxane or trisiloxane) or a silazane. Thus, for example, a composition that includes a polysiloxane according to the above described general structure, which possesses at least one Si—OH group, can be chain extended by its reaction with an alkoxy silane (for example, dialkoxy silane or trialkoxy silane) in the presence of a tin- or titanium containing catalyst.

The metal-containing catalysts in the chain extension reaction are mostly specific for a particular reaction. Such catalysts are known from the prior art and comprise, for example, metals like platinum, rhodium, tin, titanium, copper, lead, etc. In a preferred chain extension reaction, a polysiloxane having at least one aliphatic unsaturated group, preferably an end group, is reacted in the presence of a hydrosilylation catalyst with an organosilicon material that is a siloxane or polysiloxane having at least one (preferably terminal) Si—H group. The polysiloxane possesses at least one aliphatic unsaturated group and satisfies the abovementioned general formula, in which R and n are as previously defined, wherein on average, between 1 and 2 R groups per polymer possess an aliphatic unsaturated group. Representative aliphatic unsaturated groups are, for example, vinyl, allyl, hexenyl and cyclohexenyl or a group R²CH═CHR³, in which R² stands for a divalent aliphatic chain linked to the silicon and R³ stands for a hydrogen atom or an alkyl group. The organosilicon material having at least one Si—H group has preferably the above cited structure, in which R and n are as previously defined, wherein on average, between 1 and 2 R groups mean a hydrogen and n is 0 or a positive number.

This material can be a polymer or a low molecular weight material like a siloxane (for example, a disiloxane or a trisiloxane).

The polysiloxane, having at least one aliphatic unsaturated group, and the organosilicon group, having at least one Si—H group, react in the presence of a hydrosilylation catalyst. Such catalysts are known from the prior art and include, for example, platinum- and rhodium-containing materials. The catalysts can be in any known form, for example, platinum or rhodium deposited on carrier materials (for example, silica gel or active charcoal) or other suitable compounds like platinum chloride, salts of platinic acid or chloroplatinic acids. Due to its good dispersibility in organosilicon systems and to the low color changes, a preferred catalyst is chloroplatinic acid, either as the commercially available hexahydrate or in anhydrous form.

In a further preferred chain extension reaction, a polysiloxane having at least one Si—OH group, preferably an end group, is reacted with an organosilicon material that has at least one alkoxy group, preferably a siloxane having at least one Si—OR group or an alkoxy silane having at least two alkoxy groups. Again, a metal-containing catalyst is again used as the catalyst here.

For the reaction between an Si—OH group with a Si—OR group, there exist many catalysts known from the literature, for example, organometallic compounds like organotin salts, titanates or titanium chelates or complexes. Examples include tin-octoate, dibutyltin dilaurate, dibutyltin diacetate, dimethyltin dineodecanoate, dibutyltin dimethoxide, isobutyltin triceroate, dimethyltin dibutyrate, dimethyltin dineodecanoate, triethyltin tartrate, tin oleate, tin naphthenate, tin butyrate, tin acetate, tin benzoate, tin sebacate, tin succinate, tetrabutyltitanate, tetraisopropyltitanate, tetraphenyltitanate, tetraoctadecyltitanate, titanium naphthenate, ethyltriethanolamine titanate, titanium diisopropyl diethyl acetoacetate, titanium diisopropoxy diacetyl acetonate und titanium tetraalkoxide, in which the alkoxide is butoxy or propoxy.

Furthermore, the silicone-in-water emulsions preferably comprise at least one surfactant. They were described in detail above.

Likewise preferred inventive hair treating agents are characterized in that they comprise at least one silicone of formula (IV)
R₃Si—[O—SiR₂]ₓ—(CH₂)ₙ—[O—SiR₂]_{y}—O—SiR₃ (IV),
in which R stands for the same or different groups from the group —H, -phenyl, -benzyl, —CH₂—CH(CH₃)Ph, the C₁₋₂₀ alkyl groups, preferably —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, x or y stands for a number from 0 to 200, preferably from 0 to 10, more preferably from 0 to 7 and especially 0, 1, 2, CH3, 4, 5 or 6, and n stands for a number from 0 to 10, preferably from 1 to 8 and particularly for 2, 3, 4, 5, 6.

The silicones are preferably water-soluble. According to the invention, preferred hair treating agents are thus characterized in that they may further comprise a water-soluble silicone.

In a further preferred embodiment, the inventive compositions can comprise emulsifiers (F). Emulsifiers act at the interface to produce water or oil-stable adsorption layers that protect the dispersed droplets against coalescence and thereby stabilize the emulsion. Thus, emulsifiers, like surfactants are composed of hydrophobic and hydrophilic molecular moieties. Hydrophilic emulsifiers preferably form O/W emulsions and hydrophobic emulsifiers preferably form W/O emulsions. An emulsion is understood to mean a dispersion of a liquid in the form of droplets in another liquid using an energy input to afford interfaces stabilized with surfactants. The choice of this emulsifying surfactant or emulsifier depends on the materials being dispersed and the respective external phase as well as the fineness of the emulsion. Exemplary emulsifiers usable according to the invention are
Addition products of 4 to 30 moles ethylene oxide and/or 0 to 5 moles propylene oxide to linear fatty alcohols with 8 to 22 carbon atoms, to fatty acids with 12 to 22 carbon atoms and to alkyl phenols with 8 to 15 carbon atoms in the alkyl group,
C₁₂-C₂₂ fatty acid mono and diesters of addition products of 1 to 30 moles ethylene oxide to polyols with 3 to 6 carbon atoms, particularly glycerine.
ethylene oxide and polyglycerine addition products on methylglucoside fatty acid esters, fatty acid alkanolamides and fatty acid glucamides,
C₈-C₂₂ alkyl mono and oligoglycosides and their ethoxylated analogs, wherein the degrees of oligomerization are 1.1 to 5, particularly 1.2 to 2.0 and glucose is the sugar component, are preferred,
mixtures of alkyl (oligo) glucosides and fatty alcohols, for example, the commercially available product Montanov® 68,
Addition products of 5 to 60 moles ethylene oxide on castor oil and hydrogenated castor oil,
partial esters of polyols with 3-6 carbon atoms with saturated fatty acids with 8 to 22 C atoms,
Sterols. Sterols are understood to mean a group of steroids, which carry a hydroxyl group on carbon atom 3 of the steroid skeleton and are isolated from both animal tissue (zoosterols) and vegetal fats (phytosterols). Examples of zoosterols are cholesterol and lanosterol. Examples of suitable phytosterols are ergosterol, stigmasterol and sitosterol. Sterols, the so-called mycosterols, are also isolated from fungi and yeasts.
Phospholipids. Among these are principally meant the glucose-phospholipids, which are obtained e.g., as lecithins or phosphatidyl cholines from e.g., egg yolk or plant seeds (e.g., soya beans).
fatty acid esters of sugars and sugar alcohols such as sorbitol,
Polyglycerines and polyglycerine derivatives such as for example, polyglycerine poly-12-hydroxystearate (commercial product Dehymuls® PGPH),
linear and branched fatty acids with 8 to 30 C atoms and their Na, K, ammonium, Ca, Mg and Zn salts.

The inventive compositions preferably comprise the emulsifiers in quantities of 0.1 to 25 wt. %, particularly 0.5-15 wt. %, based on the total composition.

Preferably, the inventive compositions can comprise at least one non-ionic emulsifier with an HLB value of 8 to 18. Non-ionic emulsifiers with an HLB value of 10-15 can be particularly preferred according to the invention.

It has been shown to be further advantageous when polymers (G) are comprised in the inventive compositions. In a preferred embodiment, polymers are accordingly added to the inventively employed compositions, wherein not only cationic, anionic, amphoteric polymers but also non-ionic polymers have proven efficient.

Preferably, cationic or amphoteric polymers are inventively employed. Cationic or amphoteric polymers are understood to mean polymers that, in their main chain or side chain, have groups that can be “temporarily” or “permanently” cationic. “Permanently cationic” refers, according to the invention, to those polymers, which independently of the pH of the medium, have a cationic group. These are generally polymers, which comprise a quaternary nitrogen atom, in the form of an ammonium group, for example. Preferred cationic groups are quaternary ammonium groups. In particular, those polymers in which the quaternary ammonium groups are bonded through a C1-4 hydrocarbon group to a polymer backbone of acrylic acid, methacrylic acid or their derivatives, have proved to be particularly suitable.

Homopolymers of the general formula (G1-I), 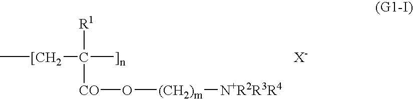
in which R¹══—H or —CH₃, R², R³ and R⁴ independently of each other are selected from C1-4 alkyl, -alkenyl or -hydroxyalkyl groups, m=1, 2, 3 or 4, n is a natural number and X⁻ is a physiologically compatible organic or inorganic anion, as well as copolymers, essentially consisting of the monomer units listed in formula (G1-I) as well as non-ionic monomer units, are particularly preferred cationic polymers. Regarding these polymers, those that are preferred in accordance with the invention meet at least one of the following conditions:
R¹ stands for a methyl group
R², R³ and R⁴ stand for methyl groups
m has the value 2.

Exemplary physiologically compatible counter ions X⁻ include halide ions, sulfate ions, phosphate ions, methosulfate ions as well as organic ions such as lactate, citrate, tartrate and acetate ions. Halide ions are preferred, particularly chloride.

A particularly suitable homopolymer is the optionally crosslinked poly(methacryloyloxyethyl trimethyl ammonium chloride) with the INCI name Polyquaternium-37. Such products are commercially available under the trade names Rheocare® CTH (Cosmetic Rheologies) and Synthalen® CR (Ethnichem). Crosslinking can be effected, when desired, with the help of olefinically polyunsaturated compounds, for example, divinylbenzene, tetraallyloxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably employed in the form of a non-aqueous polymer dispersion that should have a polymer content of not less than 30 wt. %. Such polymer dispersions are commercially available under the names Salcare® SC 95 (ca. 50% polymer content, additional components: mineral oil (INCI name: Mineral Oil) and tridecyl polyoxypropylene polyoxyethylene ether (INCI name: PPG-1-Trideceth-6)) and Salcare® SC 96 (ca. 50% polymer content, additional components: mixture of diesters of propylene glycol with a mixture of caprylic- and capric acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (G1-I) preferably comprise acrylamide, methacrylamide, C₁₄ alkyl esters of acrylic acid and C₁₄ alkyl esters of methacrylic acid as the non-ionic monomer units. Acrylamide is particularly preferred among these non-ionic monomers. These copolymers can also be crosslinked, as in the case of the above described homopolymers. An inventively preferred copolymer is the crosslinked acrylamide/methacryloyloxyethyl trimethyl ammonium chloride copolymer. Such copolymers, in which the monomers are present in a weight ratio of about 20:80, are commercially available as a ca. 50% conc. non-aqueous polymer dispersion under the trade name Salcare® SC 92.

Further preferred cationic polymers are, for example:
quaternized cellulose derivatives, commercially available under the trade names Celquat® and Polymer JR®. The compounds Celquat® H 100, Celquat® L 200 and Polymer JF®400 are preferred quaternized cellulose derivatives,
cationic alkyl polyglycosides according to DE-PS 44 13 686,
cationized honey, for example, the commercial product Honeyquat® 50, cationic guar derivatives, such as in particular, the products marketed under the trade names Cosmedia® Guar and Jaguar,
polymeric dimethyl diallyl ammonium salts and their copolymers with esters and amides of acrylic acid and methacrylic acid. The commercially available products Merquat®100 (poly(dimethyl diallyl ammonium chloride)) and Merquat®550 (dimethyl diallyl ammonium chloride-acrylamide copolymer) are examples of such cationic polymers.
copolymers of vinyl pyrrolidone with quaternized derivatives of dialkylamino acrylate and dialkylamino methacrylate, such as, for example, vinyl pyrrolidone-dimethylaminoethyl methacrylate copolymers quaternized with diethyl sulfate. Such compounds are commercially available under the trade names Gafquat®734 and Gafquat®755.
Vinyl pyrrolidone-vinylimidazolium methochloride copolymers, as are offered under the trade names Luviquat FC 370, FC 550, FC 905, HM 552,
quaternized polyvinyl alcohol,
as well as the known polymers containing quaternary nitrogen atoms in the main polymer chain, Polyquaternium 2, Polyquaternium 17, Polyquaternium 18 and Polyquaternium 27.

Polymers designated as Polyquaternium-24 (commercial product e.g., Quatrisoft® LM 200) can also be employed as cationic polymers. The copolymers of vinyl pyrrolidone are also usable according to the invention, such as the commercially available products Copolymer845 (manufacturer: ISP), Gaffix®VC 713 (manufacturer: ISP), Gafquat®ASCP 1011, Gafquat®HS 110, Luviquat®8155 and Luviquat® MS 370.

Further cationic polymers that can be employed in the inventive compositions are the “temporarily cationic” polymers. These polymers usually comprise an amino group that is present at specific pH values as the quaternary ammonium group and is thus cationic. Chitosan and its derivatives, such as for example, the commercially available Hydagen® CMF, Hydagen® HCMF, Kytamer® PC and Chitolam® NB/101 are preferred.

Inventively preferred cationic polymers are cationic cellulose derivatives and chitosan and its derivatives, in particular, the commercial products Polymer® JR 400, Hydagen® HCMF and Kytamer® PC, cationic guar derivatives, cationic honey derivatives, in particular, the commercial product Honeyquat® 50, cationic alkyl polyglycosides according to DE-PS 44 13 686 and polymers of the type Polyquaternium-37.

In addition, cationized protein hydrolyzates are considered as cationic polymers, wherein the basic protein hydrolyzate can originate from animals, for example, from collagen, milk or keratin, from plants, for example, from wheat, maize, rice, potatoes, soya or almonds, from marine life, for example, from fish collagen or algae, or from biotechnologically obtained protein hydrolyzates. The inventive cationic derivatives based on protein hydrolyzates can be obtained from the corresponding proteins by a chemical, particularly alkaline or acid hydrolysis, by an enzymatic hydrolysis and/or a combination of both types of hydrolysis. The hydrolysis of proteins generally produces a protein hydrolyzate with a molecular weight distribution from about 100 daltons up to several thousand daltons. Cationic protein hydrolyzates are preferred, whose base protein content has a molecular weight of 100 to 25,000 daltons, preferably 250 to 5,000 daltons. Moreover, cationic protein hydrolyzates are understood to include quaternized amino acids and their mixtures. Quaternization of the protein hydrolyzates or the amino acids is often carried out using quaternary ammonium salts such as, for example, N,N-dimethyl-N(n-alkyl)-N-(2-hydroxy-3-chloro-n-propyl) ammonium halides. Moreover, the cationic protein hydrolyzates can also be further derivatized. Typical examples of inventive cationic protein hydrolyzates and derivatives are the commercially available products and those cited under the INCI names in the “International Cosmetic Ingredient Dictionary and Handbook,” (seventh edition 1997, The Cosmetic, Toiletry, and Fragrance Association 1101 17^{th} Street, N. W., Suite 300, Washington, DC 20036-4702): Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein.

The cationic protein hydrolyzates and derivatives based on plants are quite particularly preferred.

In addition to cationic polymers, or in their place, the inventive compositions can also comprise amphoteric polymers. In addition they have at least one negatively charged group in the molecule and are also called zwitterionic polymers. In the context of the present invention, preferred employable zwitterionic polymers are essentially composed of

A) Monomers with quaternary ammonium groups of the general formula (Z-I),
R¹—CH═CR²—CO—Z—(CₙH₂ₙ)—N(+)R³R⁴R⁵ A⁽⁻⁾ (Z-I)
in which R¹ and R² independently of each other stand for hydrogen or a methyl group and R³, R⁴ and R⁵ independently of one another for alkyl groups with 1 to 4 carbon atoms, Z for an NH-group or an oxygen atom, n for a whole number from 2 to 5 and A⁽⁻⁾ is the anion of an organic or inorganic acid and

B) monomeric carboxylic acids of the general formula (Z-II),
R⁶—CH═CR⁷—COOH (II)
in which R⁶ and R⁷, independently of one another are hydrogen or methyl groups.

Suitable starting monomers are e.g., dimethylaminoethyl acrylamide, dimethylaminoethyl methacrylamide, dimethylaminopropyl acrylamide, dimethylaminopropyl methacrylamide and diethylaminoethyl acrylamide, when Z means an NH group, or dimethylaminoethyl acrylate, dimethylaminoethyl methacrylate and diethylaminoethyl acrylate when Z is an oxygen atom.

The monomers comprising a tertiary amino group are then quaternized in the usual manner, wherein methyl chloride, dimethyl sulfate or diethyl sulfate are particularly suitable as the alkylation reagents. The quaternization reaction can be made in aqueous solution or in a solvent.

Advantageously, those monomers of formula (Z-I) are used, which are derivatives of acrylamide or methacrylamide. In addition, those monomers that comprise halide, methoxysulfate or ethoxysulfate ions as the counter ions are preferred. Those monomers of formula (Z-I), in which R³, R⁴ and R⁵ are methyl groups, are likewise preferred.

Acrylamidopropyl trimethyl ammonium chloride is a quite particularly preferred monomer of formula (Z-I).

Acrylic acid, methacrylic acid, crotonic acid and 2-methylcrotonic acid are suitable as the monomeric carboxylic acid of formula (Z-II). Acrylic acid or methacrylic acid, in particular, acrylic acid, is preferably employed.

The inventively employable zwitterionic polymers are manufactured from monomers of Formulas (Z-I) and (Z-II) according to known polymerization processes. The polymerization can be made in either aqueous or aqueous alcoholic solution. Alcohols containing 1 to 4 carbon atoms, preferably isopropanol, can be used as the alcohols, which simultaneously act as polymerization regulators. However, other components can also be added to the monomer solution, e.g., formic acid or mercaptans, such as thioethanol and thioglycolic acid. The polymerization is initiated by means of radical-forming substances. For this, redox systems and/or thermally decomposing radical sources of the azo compound type, such as e.g., azoisobutyric acid nitrile, azobis(cyclopentanoic acid) or azobis(amidinopropane) dihydrochloride, can be used. Combinations of hydrogen peroxide, potassium or ammonium peroxodisulfate as well as tertiary butyl hydroperoxide are suitable redox systems, with sodium sulfite, sodium dithionite or hydroxylamine hydrochloride as the reductive components.

The polymerization can be carried out isothermally or under adiabatic conditions, wherein, depending on the concentration ratios, the temperature range for the reaction process can vary between 20 and 200° C. due to the heat of reaction of polymerization, and the reaction possibly has to be carried out under the resulting overpressure. Preferably, the reaction temperature is between 20 and 100° C.

During the copolymerization, the pH can vary over a wide range. Advantageously, polymerization is carried out at low pH; however, pH values above the neutral point are also possible. After the polymerization, the pH is adjusted to between 5 and 10, preferably 6 to 8, with an aqueous base, e.g., sodium hydroxide, potassium hydroxide or ammonium hydroxide. Further details of the polymerization process can be found in the examples.

Those polymers that possess an excess of monomers of formula (Z-I) over the monomers of formula (Z-II) have proved to be particularly effective. Accordingly, it is inventively preferred to use such polymers that consist of monomers of formula (Z-I) and monomers of formula (Z-II) in a molar ratio of 60:40 to 95:5, particularly 75:25 to 95:5.

The inventive compositions preferably comprise the cationic or amphoteric polymers in quantities of 0.05 to 10 wt. %, based on the total composition. Quantities of 0.1 to 5 wt. % are particularly preferred.

The anionic polymers (G2) concern anionic polymers that possess carboxylate and/or sulfonate groups. Exemplary anionic monomers, from which such polymers can be made, are acrylic acid, methacrylic acid, crotonic acid, maleic anhydride and 2-acrylamido-2-methylpropane sulfonic acid. Here, the acidic groups may be fully or partially present as sodium, potassium, ammonium, mono- or triethanol ammonium salts. Preferred monomers are 2-acrylamido-2-methylpropane sulfonic acid and acrylic acid.

Anionic polymers that comprise 2-acrylamido-2-methylpropane sulfonic acid alone or as the comonomer, have proven to be quite particularly effective; the sulfonic acid group may be fully or partially present as the sodium, potassium, ammonium, mono- or triethanol ammonium salt.

The homopolymer of 2-acrylamido-2-methylpropane sulfonic acid, which is commercially available, for example, under the trade name Rheothik®11-80, is particularly preferred.

In this embodiment, it can be preferred to use copolymers of at least one anionic monomer and at least one non-ionic monomer. Regarding the anionic monomers, reference is made to the abovementioned substances. Preferred non-ionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers and particularly polyacrylamide copolymers with monomers that contain sulfonic acid groups. A particularly preferred anionic copolymer consists of 70 to 55 mole% acrylamide and 30 to 45 mole% 2-acrylamido-2-methylpropane sulfonic acid, wherein the sulfonic acid group may be fully or partially present as the sodium, potassium, ammonium, mono- or triethanol ammonium salt. This copolymer can also be crosslinked, wherein the preferred crosslinking agents include polyolefinic unsaturated compounds such as tetraallyloxyethane, allylsucrose, allylpentaerythritol and methylene bisacrylamide. Such a polymer is comprised in the commercial product Sepigel®305 from the SEPPIC Company. The use of this compound, which comprises a mixture of hydrocarbons (C₁₃-C₁₄ isoparaffins) and a non-ionic emulsifier (Laureth-7) besides the polymer components, has proved to be particularly advantageous in the context of the inventive teaching.

The sodium acryloyl dimethyl taurate copolymers commercialized as a compound with isohexadecane and polysorbate 80, under the trade name Simulgel®600, have also proved to be particularly effective according to the invention.

Likewise preferred anionic homopolymers are uncrosslinked and crosslinked polyacrylic acids. Here, the preferred crosslinking agents can be allyl ethers of pentaerythritol, of sucrose and of propylene. Such compounds are commercially available under the trade name Carbopol®, for example.

Copolymers of maleic anhydride and methyl vinyl ether, especially those with crosslinks are also color-conserving polymers. A maleic acid-methyl vinyl ether copolymer, crosslinked with 1,9-decadiene, is commercially available under the name Stabileze® QM.

In addition, amphoteric polymers (G3) can be used as polymers to increase the action of the inventive active substance complex (A). The term amphopolymers embraces not only those polymers, whose molecule includes both free amino groups and free —COOH or SO₃H groups and which are capable of forming inner salts, but also zwitterionic polymers whose molecule comprises quaternary ammonium groups and —COO⁻ or —SO₃⁻ groups, and polymers comprising —COOH or SO₃H groups and quaternary ammonium groups.

An example of an amphopolymer which may be used in accordance with the invention is the acrylic resin obtainable under the designation Amphomer®, which constitutes a copolymer of tert-butylaminoethyl methacrylate, N-(1,1,3,3-tetramethylbutyl)acrylamide, and two or more monomers from the group consisting of acrylic acid, methacrylic acid and their simple esters.

Preferably employed amphoteric polymers are such polymers that are essentially composed from
(a) Monomers with quaternary ammonium groups of the general formula (G3-I),
   R¹—CH═CR²—CO—Z—(CₙH₂ₙ)—N⁽⁺⁾R³R⁴R⁵ A⁽⁻⁾ (G3-I)
   in which R¹ and R² independently of each other stand for hydrogen or a methyl group and R³, R⁴ and R⁵ independently of one another for alkyl groups with 1 to 4 carbon atoms, Z for an NH-group or an oxygen atom, n for a whole number from 2 to 5 and A⁽⁻⁾ is the anion of an organic or inorganic acid, and
(b) monomers of carboxylic acids of the general formula (G3-II),
   R⁶—CH═CR⁷—COOH (G3-II)
   in which R⁶ and R⁷, independently of one another are hydrogen or methyl groups.

According to the invention, these compounds can be both added directly as well as in salt form, which is obtained by neutralization of the polymer with an alkali hydroxide, for example. Quite particularly preferred are such polymers, which incorporate monomers of type (a), in which R³, R⁴ and R⁵ are methyl groups, Z is an NH-group and A⁽⁻⁾ is a halide, methoxysulfate or ethoxysulfate ion; acrylamidopropyl trimethyl ammonium chloride is a particularly preferred monomer (a). Acrylic acid is preferably used as the monomer (b) in the cited polymers.

In a further variant, the inventive compositions can additionally comprise non-ionic polymers (G4).

Suitable non-ionic polymers are, for example:
vinyl pyrrolidone-vinyl ester copolymers, such as, for example, those marketed by BASF under the trade name Luviskol®, Luviskol® VA 64 and Luviskol® VA 73, each vinyl pyrrolidone-vinyl acetate copolymers, are likewise preferred non-ionic polymers.
Cellulose ethers, such as hydroxypropyl cellulose, hydroxyethyl cellulose, and methyl hydroxypropyl cellulose, as marketed for example, under the trademarks Culminal® and Benecel® (AQUALON) and Natrosol® types (Hercules).
Starch and its derivatives, especially starch ethers, for example, Structure® XL (National Starch), a multifunctional, salt tolerant starch;
   Shellac
Polyvinyl pyrrolidones, as are marketed, for example, under the designation Luviskol® (BASF).
Siloxanes. These siloxanes can be both water-soluble and also water-insoluble. Both volatile and non-volatile siloxanes are suitable, whereby non-volatile siloxanes are understood to mean such compounds with a boiling point above 200° C. at normal pressure. Preferred siloxanes are polydialkylsiloxanes, such as, for example, polydimethylsiloxane, polyalkylarylsiloxanes, such as, for example, polyphenylmethylsiloxane, ethoxylated polydialkylsiloxanes as well as polydialkylsiloxanes, which comprise amine and/or hydroxyl groups.
Glycosidically substituted silicones.

According to the invention, it is also possible for the preparations used to comprise a plurality, particularly two different polymers of the same charge and/or each with an anionic and an amphoteric and/or non-ionic polymer.

The compositions used according to the invention preferably comprise the polymers (G) in quantities of 0.05 to 10 wt. %, based on the total composition. Quantities of 0.1 to 5 wt. %, particularly 0.1 to 3 wt. %, are particularly preferred.

In addition to the cited substances, the inventive compositions can comprise additional care materials. Particularly preferably, these are, for example, vitamins, provitamins or vitamin precursors, such that inventively preferred compositions are those comprising additionally at least one material from the group of vitamins, provitamins and vitamin precursors as well as their derivatives, wherein vitamins, provitamins and vitamin precursors are preferred, which are classified in the groups A, B, C, E, F and H. They were described in detail above.

A further group of care materials that can be comprised in the inventive compositions are the protein hydrolyzates and their derivatives (P). Protein hydrolyzates are product mixtures obtained by acid-, base- or enzyme-catalyzed degradation of proteins (albumins). According to the invention, the term “protein hydrolyzates” is also understood to mean total hydrolyzates as well as individual amino acids and their derivatives as well as mixtures of different amino acids. Furthermore, according to the invention, polymers built up from amino acids and amino acid derivatives are understood to be included in the term protein hydrolyzates. The latter include for example, polyalanine, polyasparagine, polyserine etc. Additional examples of usable compounds according to the invention are L-alanyl-L-proline, polyglycine, glycyl-L-glutamine or D/L-methionine-S-methyl sulfonium chloride. Of course, β-amino acids and their derivatives like β-alanine, anthranilic acid or hippuric acid can also be added according to the invention. The molecular weight of the inventive usable protein hydrolyzates ranges between 75, the molecular weight of glycine, and 200,000, preferably the molecular weight is 75 to 50,000 and quite particularly preferably 75 to 20,000 dalton.

According to the invention, the added protein hydrolyzates can be of both vegetal as well as animal or marine or synthetic origin.

Animal protein hydrolyzates are, for example, elastin, collagen, keratin and milk protein hydrolyzates, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

According to the invention, it is preferred to use protein hydrolyzates of vegetal origin, e.g., soya-, almond-, pea-, potato- and wheat protein hydrolyzates. Such products are available, for example, under the trade names Gluadin® (Cognis), DiaMin® (Diamalt), Lexein® (Inolex) Hydrosoy® (Croda), Hydrolupin® (Croda), Hydrosesame® (Croda), Hydrotritium® (Croda) and Crotein® (Croda).

Although it is preferred to add the protein hydrolyzates as such, optionally other mixtures containing amino acids can also be added in their place Likewise, it is possible to add derivatives of protein hydrolyzates, e.g., in the form of their fatty acid condensation products. Such products are marketed, for example, under the trade names Lamepon® (Cognis), Lexein® (Inolex), Crolastin® (Croda) or Crotein® (Croda).

Naturally, the inventive teaching includes all isomeric forms, such as cis trans isomers, diastereoisomers and chiral isomers.

According to the invention, it is also possible to employ a mixture of a plurality of protein hydrolyzates (P).

The compositions comprise the protein hydrolyzates (P) in concentrations of 0.01 wt. % to 20 wt. %, preferably 0.05 wt. % up to 15 wt. % and quite particularly preferably in amounts of 0.05 wt. % up to 5 wt. %.

Moreover, in a preferred embodiment of the invention, an inventive composition can also comprise UV filters (I). The inventively useable UV filters are not generally limited in regard to their structure and their physical properties. Indeed, all UV filters that can be employed in the cosmetic field having an absorption maximum in the UVA (315-400 nm), in the UVB (280-315 nm) or in the UVC (<280 nm) regions are suitable. UV filters having an absorption maximum in the UVB region, especially in the range from about 280 to about 300 nm, are particularly preferred.

The UV-filters used in the context of the invention are chosen from substituted benzophenones, p-aminobenzoates, diphenylacrylates, cinnamates, salicylates, benzimidazoles and o-aminobenzoates.

Exemplary inventively useable UV filters are 4-aminobenzoic acid, N,N,N-trimethyl-4-(2-oxoborn-3-ylidenemethyl)aniline methyl sulfate, 3,3,5-trimethylcyclohexyl salicylate (Homosalate), 2-hydroxy-4-methoxybenzophenone (Benzophenone-3; Uvinul®M 40, Uvasorb®MET, Neo Heliopan®BB, Eusolex®4360), 2-phenylbenzimidazol-5-sulfonic acid and its potassium, sodium and triethanolamine salts (Phenylbenzimidazole sulfonic acid; Parsol®HS; Neo Heliopan®Hydro), 3,3′-(1,4-phenylenedimethylene)-bis(7,7-dimethyl-2-oxo-bicyclo-[2.2.1]hept-1-yl-methane sulfonic acid) and its salts, 1-(4-tert.-butylphenyl)-3-(4-methoxyphenyl)-propane-1,3-dione (Butyl methoxydibenzoylmethane; Parsol®1789, Eusolex®9020), α-(2-oxoborn-3-ylidene)-toluene-4-sulfonic acid and its salts, ethoxylated 4-aminobenzoic acid ethyl ester (PEG-25 PABA; Uvinul®P 25), 4-dimethylaminobenzoic acid 2-ethylhexyl ester (Octyl Dimethyl PABA; Uvasorb®DMO, Escalol®507, Eusolex®6007), salicylic acid 2-ethylhexyl ester (Octyl Salicylat; Escalol®587, Neo Heliopan®OS, Uvinul®O18), 4-methoxycinnamic acid isopentyl ester (Isoamyl p-Methoxycinnamate; Neo Heliopan®E 1000), 4-methoxycinnamic acid 2- ethylhexyl ester (Octyl Methoxycinnamate; Parsol®MCX, Escalol®557, Neo Heliopan®AV), 2-hydroxy-4-methoxybenzophenone-5-sulfonic acid and its sodium salt (Benzophenone-4; Uvinul®MS 40; Uvasorb®S 5), 3-(4′-methylbenzylidene)-D,L-camphor (4-Methylbenzylidene camphor; Parsol®5000, Eusolex®6300), 3-benzylidene camphor (3-Benzylidene camphor), 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, polymers of N-{(2 and 4)-[2-oxoborn-3-ylidenemethyl]benzyl}-acrylamide, 2,4-dihydroxybenzophenone (Benzophenone-1; Uvasorb®20 H, Uvinul®400), 1,1′-diphenylacrylonitrile acid 2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid 2′-ethylhexyl ester. 4-Amino-benzoic acid, N,N,N-trimethyl-4-(2-oxoborn-3-ylidenemethyl)aniline methyl sulfate, 3,3,5-trimethylcyclohexyl salicylate, 2-hydroxy-4-methoxy-benzophenone, 2-phenylbenzimidazole-5-sulfonic acid and its potassium, sodium and triethanolamine salts, 3,3′-(1,4-phenylenedimethylene)-bis(7,7-dimethyl-2-oxo-bicyclo-[2.2.1]hept-1-yl-methane sulfonic acid) and its salts, 1-(4-tert.-butylphenyl)-3-(4-methoxyphenyl)-propane-1,3-dione, α-(2-oxoborn-3-ylidene)-toluene-4-sulfonic acid and its salts, ethoxylated 4-aminobenzoic acid ethyl ester, 4-dimethylaminobenzoic acid 2- ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and its sodium salt, 3-(4′-methylbenzylidene)-D,L-camphor, 3-benzylidene camphor, 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, polymers of N-{(2 and 4)-[2-oxoborn-3-ylidenemethyl]benzyl} acrylamide are preferred. According to the invention, 2-hydroxy-4-methoxy-benzophenone, 2-phenylbenzimidazole-5-sulfonic acid and its potassium, sodium and triethanolamine salts, 1-(4-tert.-butylphenyl)-3-(4-methoxyphenyl)-propane-1,3-dione, 4-methoxycinnamic acid 2-ethylhexyl ester and 3-(4′-methylbenzylidene)-D,L-camphor are quite particularly preferred.

Those UV filters with a molecular extinction coefficient at the absorption maximum of above 15,000, particularly 20,000, are preferred.

Moreover, it was found that for structurally similar UV filters, in many cases in the context of the inventive teaching, the water-insoluble compound exhibits a higher activity than that of water-soluble compounds that differ from them by one or a plurality of additional ionic groups. In the context of the invention, water-insoluble UV filters are understood to mean those that dissolve not more than 1 wt. %, especially not more than 0.1 wt. % in water at 20° C. In addition, these compounds should be soluble to at least 0.1, especially to at least 1 wt. % in conventional cosmetic oil components at room temperature. Accordingly, the use of water-insoluble UV filters can be inventively preferred.

According to a further embodiment of the invention, those UV filters are preferred, which have a cationic group, especially a quaternary ammonium group.

These UV filters have the general structure U-Q.

Here, the structural component U stands for a group that absorbs UV radiation. In principle, these groups can derive from the known abovementioned UV filters that can be employed in the field of cosmetics, in which one group, generally a hydrogen atom of the UV filter is replaced by a cationic group Q, in particular, with a quaternary amino function.

Compounds, from which the structural component U can be derived are, for example:
substituted benzophenones,
p-aminobenzoic acid esters,
diphenylacrylic acid esters,
cinnamic acid esters,
salicylic acid esters,
benzimidazoles and
o-aminobenzoic acid esters.

Structural components U that derive from cinnamic acid amide or from N,N-dimethylaminobenzoic acid amide are inventively preferred.

In principle, the structural components U can be selected such that the absorption maximum of the UV filter can be in the UVA (315-400 nm) region, as well as in the UVB(280-315 nm) region or in the UVC(<280 nm) region. UV filters having an absorption maximum in the UVB region, especially in the range from about 280 to about 300 nm, are particularly preferred.

Furthermore, the structural component U, also depending on the structural component Q, is selected such that the molar extinction coefficient of the UV filter at the absorption maximum is above 15,000, especially above 20,000.

The structural component Q preferably comprises a quaternary ammonium group as the cationic group. In principle, this quaternary ammonium group can be directly bonded to the structural component U, such that the structural component U represents one of the four substituents of the positively charged nitrogen atom. Preferably however, one of the four substituents on the positively charged nitrogen atom is a group, in particular, an alkyl group containing 2 to 6 carbon atoms, which acts as the link between the structural component U and the positively charged nitrogen atom.

Advantageously, the group Q has the general structure —(CH₂)ₓ—N⁺R¹R²R³ X⁻, in which x stands for an integer from 1 to 4, R¹ and R² independently of one another stand for C₁₋₄ alkyl groups, R³ stands for a C₁₋₂₂ alkyl group or a benzyl group and X⁻ for a physiologically compatible anion. In the context of this general structure, x preferably stands for the number 3, R¹ and R² each for a methyl group and R³ either for a methyl group or a saturated or unsaturated, linear or branched hydrocarbon chain containing 8 to 22, particularly 10 to 18 carbon atoms.

Exemplary physiologically compatible anions are inorganic anions such as halides, particularly chloride, bromide and fluoride, sulfate ions and phosphate ions as well as organic anions such as lactate, citrate, acetate, tartrate, methosulfate and tosylate.

Two preferred UV filters containing cationic groups are the compounds cinnamic acid amidopropyl trimethyl ammonium chloride (Incroquat® UV-283) and dodecyl dimethylaminobenzamidopropyl dimethyl ammonium tosylate (Escalol® HP 610).

Of course, the inventive teaching also includes the use of a combination of a plurality of UV filters. In the context of this embodiment, the combination of at least one water-insoluble UV filter with at least one UV filter containing a cationic group is preferred.

The compositions used according to the invention preferably comprise the UV filters in quantities of 0.01 to 5 wt. %, based on the total composition. Quantities of 0.4-2.5 wt. % are preferred.

The inventive compositions can further comprise a 2-pyrrolidone-5-carboxylic acid and derivatives (J) thereof. The sodium, potassium, calcium, magnesium or ammonium salts are preferred, in which the ammonium ion carries one to three C₁- to C₄ alkyl groups besides hydrogen. The sodium salt is quite particularly preferred. The quantities employed in the inventive compositions preferably range from 0.05 to 10 wt. %, based on the total composition, particularly preferably 0.1 to 5, and particularly 0.1 to 3 wt. %.

Finally, the inventive composition can also comprise plant extracts (L).

Usually, these extracts are manufactured by extraction of the whole plant. In individual cases, however, it can also be preferred to manufacture the extracts solely from blossoms and/or leaves of the plant.

With regard to the inventively usable plant extracts, we particularly refer to extracts that are listed in the Table beginning on page 44 of the 3rd edition of the Guidelines for the Declaration of Ingredients in Cosmetics, (Leitfadens zur Inhaltsstoffdeklaration kosmetischer Mittel) published by the German Cosmetics, Toiletry, Perfumery and Detergent Association e.V. (IKW), Frankfurt.

According to the invention, mainly extracts from green tea, oak bark, stinging nettle, hamamelis, hops, henna, camomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger are preferred.

Extracts from green tea, oak bark, stinging nettle, hamamelis, hops, camomile, burdock root, hawthorn, linden flowers, almonds, aloe vera, coconut, mango, apricot, lime, wheat, kiwi, melon, orange, grapefruit, sage, rosemary, birch, lady's smock, common yarrow, rest-harrow, meristem, ginseng and ginger are preferred.

Extracts of green tea, almonds, aloe vera, coconut, mango, apricot, lime, wheat, kiwi and melon are quite particularly suitable for the inventive use.

The extracting agent used to prepare the cited plant extracts can be water, alcohols as well as their mixtures. Exemplary preferred alcohols are lower alcohols such as ethanol and isopropanol, but particularly polyhydroxy alcohols such as ethylene glycol, propylene glycol and butylene glycol, both as the sole extracting agent as well as in aqueous mixtures. Plant extracts based on water/propylene glycol in the ratio 1:10 to 10:1 have proven particularly suitable.

According to the invention, the plant extracts can be used in pure and also in diluted form. When they are used in diluted form, they normally comprise ca. 2-80 wt. % active substance and the solvent is the extracting agent or mixture of extracting agents used for their preparation.

In addition, it can be preferred to add mixtures of a plurality, particularly two different plant extracts to the inventive agent.

In addition it can prove advantageous when the inventive compositions comprise penetration aids and/or swelling agents (M). These include for example, urea and urea derivatives, guanidine and its derivatives, arginine and its derivatives, water glass, imidazole and its derivatives, histidine and its derivatives, benzyl alcohol, glycerine, glycol and glycol ethers, propylene glycol and propylene glycol ethers, for example, propylene glycol monoethyl ether, carbonates, hydrogen carbonates, diols and triols, and particularly 1,2-diols and 1,3-diols such as for example, 1,2-propane diol, 1,2-pentane diol, 1,2-hexane diol, 1,2-dodecane diol, 1,3-propane diol, 1,6-hexane diol, 1,5-pentane diol, 1,4-butane diol.

In the context of the invention short chain carboxylic acids (N) can, in addition, advantageously support the complex of active substances (A). In the context of the invention, short chain carboxylic acids and their derivatives are understood to mean carboxylic acids that can be saturated or unsaturated and/or linear or branched or cyclic and/or aromatic and/or heterocyclic and have a molecular weight of less than 750. In the context of the invention, saturated or unsaturated or linear or branched carboxylic acids with a chain length of 1 to 16 carbon atoms in the chain can be preferred, those with a chain length of 1 up to 12 carbon atoms in the chain are quite particularly preferred.

In the context of the invention, the short chain carboxylic acids can have one, two, three or more carboxyl groups. In the context of the invention, carboxylic acids with a plurality of carboxyl groups are preferred, particularly di and tricarboxylic acids. The carboxyl groups can be totally or partially present as esters, acid anhydrides, lactones, amides, imide acid, lactams, lactims, dicarboximides, carbohydrazide, hydrazone, hydroxams, hydroxims, amidines, amidoximes, nitriles, phosphon- or phosphate esters. The inventive carboxylic acids can of course be substituted along the carbon chain or on the cyclic structure. The substituents of the inventive carboxylic acids include, for example, C1-C8-alkyl-, C2-C8-alkenyl-, aryl-, aralkyl- and aralkenyl-, hydroxymethyl-, C2-C8-hydroxyalkyl-, C2-C8-hydroxyalkenyl-, aminomethyl-, C2-C8-aminoalkyl-, cyano-, formyl-, oxo-, thioxo-, hydroxy-, mercapto-, amino-, carboxyl- or imino groups. Preferred substituents are C1-C8-alkyl-, hydroxymethyl-, hydroxy-, amino- and carboxyl groups. Substituents in the a-position are particularly preferred. Quite particularly preferred substituents are hydroxy-, alkoxy- and amino groups, wherein the amino function can be optionally further substituted by alkyl, aryl, aralkyl and/or alkenyl groups. In addition, equally preferred carboxylic acid derivatives are the phosphonate- and phosphate esters.

Exemplary inventive carboxylic acids are formic acid acetic acid, propionic acid, butyric acid, isobutyric acid, valerianic acid, isovalerianic acid, pivalic acid, oxalic acid, malonic acid, succinic acid, glutaric acid, glycerinic acid, glyoxylic acid, adipic acid, pimelic acid, cork acid, azelaic acid, sebacic acid, propiolic acid, crotonic acid, isocrotonic acid, elaidic acid, maleic acid, fumaric acid, muconic acid, citraconic acid, mesaconic acid, campheric acid, benzoic acid, o,m,p-phthalic acid, naphthoic acid, toluoylic acid, hydratropic acid, atropic acid, cinnamic acid, isonicotinic acid, nicotinic acid, bicarbaminic acid, 4,4′-dicyano-6,6′-binicotinic acid, 8-carbamoyloctanoic acid, 1,2,4-pentanetricarboxylic acid, 2-pyrrolcarboxylic acid, 1,2,4,6,7-napthalenepentaacetic acid, malonaldehydic acid, 4-hydroxy-phthalamidic acid, 1-pyrazolecarboxylic acid, gallic acid or propanetricarboxylic acid, a dicarboxylic acid selected from the group made up of compounds of the general formula (N-I), 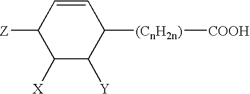
(N-I)
in which Z stands for a linear or branched alkyl or alkenyl radical containing 4 to 12 carbon atoms, n for a number from 4 to 12 as well as one of both the groups X and Y for a COOH group and the other for hydrogen or a methyl or ethyl group, dicarboxylic acids of the general formula (N-I), which additionally have 1 to 3 methyl or ethyl substituents on the cyclohexene ring as well as dicarboxylic acids that are obtained from the dicarboxylic acids according to formula (N-I) by the formal addition of a molecule of water on the double bond in the cyclohexene ring.

Dicarboxylic acids of formula (N-I) are known in the literature.

The dicarboxylic acids of formula (N-I) can be manufactured for example, by a Diels-Alder cyclization by reacting polyunsaturated carboxylic acids with unsaturated monocarboxylic acids. Usually, a polyunsaturated fatty acid is the starting material for the dicarboxylic acid component. Linoleic acid obtained from natural fats and oils is preferred. Acrylic acid, but also e.g., methacrylic acid and crotonic acid are particularly preferred as the monocarboxylic acid. Diels-Alder reactions usually result in mixtures of isomers, in which one component is in excess. According to the invention, this mixture of isomers can be added just like the pure compound.

According to the invention, besides the preferred dicarboxylic acids according to formula (N-I), other such dicarboxylic acids can be added; they differ from the compounds of formula (N-I) by the 1 to 3 methyl or ethyl substituents on the cyclohexene ring or are formed from these compounds by the formal addition of one molecule water on the double bond of the cyclohexene ring.

The dicarboxylic acid (mixture) that results from the reaction of linoleic acid with acrylic acid has proven to be particularly inventively advantageous. It is a mixture of 5- and 6-carboxy-4-hexyl-2-cyclohexene-1-octanoic acid. Such compounds are commercially obtainable under the trade names Westvaco Diacid® 1550 and Westvaco Diacid® 1595 (Manufacturer: Westvaco).

Besides the exemplary previously listed short chain carboxylic acids themselves, their physiologically acceptable salts can also be added according to the invention. Examples of such salts are the alkali- alkaline earth-, zinc salts as well as ammonium salts, under which in the context of the present invention are also understood to mean the mono-, di- and trimethyl-, -ethyl and -hydroxyethyl ammonium salts. However, with alkaline reacting amino acids such as for example, arginine, lysine, ornithine and histidine, in the context of the invention, it is quite particularly preferred to be able to add neutralized acids. Moreover, on formulation grounds, it can be preferred to select the carboxylic acid from the water-soluble representatives, in particular, the water-soluble salts.

In addition, it is inventively preferred to add hydroxycarboxylic acids with the active substance (A), and here once again particularly the dihydroxy-, trihydroxy- and polyhydroxy carboxylic acids as well as the dihydroxy-, trihydroxy- and polyhydroxy di-, tri- and polycarboxylic acids. In this respect, it was shown that besides the hydroxycarboxylic acids, also the hydroxycarboxylic acid esters as well as mixtures of hydroxycarboxylic acids and their esters and also polymeric hydroxycarboxylic acids and their esters can be quite particularly preferred. Preferred hydroxycarboxylic acid esters are fully esterified glycolic acid, lactic acid, malic acid, tartaric acid or citric acid, for example. Additional fundamentally suitable hydroxycarboxylic acid esters are esters of β-hydroxypropionic acid, of tartronic acid, of D-gluconic acid, of saccharic acid, of mucic acid or of glucuronic acid. Primary, linear or branched aliphatic alcohols with 8-22 carbon atoms, i.e., fatty alcohols or synthetic fatty alcohols, are suitable alcohol moieties of these esters. Esters of C12-C15 fatty alcohols are particularly preferred in this respect. Esters of this type are commercially available, e.g., under the trade name Cosmacol® from Enichem, Augusta Industriale. Particularly preferred polyhydroxypolycarboxylic acids are polylactic acid and polytartaric acid as well as their esters.

Further subject matters of the present invention are the use of corneocyte proteins or corneocyte polypeptides with a molecular weight of 10 to 40 kDa for improving at least one of the properties
tensile strength of keratinic fibers, especially human hair;
stabilization of the moisture balance of keratinic fibers, especially human hair;
combability of keratinic fibers, especially human hair;
slowing down the aging process of keratinic fibers, especially human hair;
restructurability of keratinic fibers, especially human hair, during and after the permanent waving process;
reducing the fall-off in elasticity of keratinic fibers, especially human hair, from damage by atmospheric action;
   as well as the use of inventive hair treating agents for improving at least one of the properties
tensile strength of keratinic fibers, especially human hair;
stabilization of the moisture balance of keratinic fibers, especially human hair;
combability of keratinic fibers, especially human hair;
slowing down the aging process of keratinic fibers, especially human hair;
restructurability of keratinic fibers, especially human hair, during and after the permanent waving process;
reducing the fall-off in elasticity of keratinic fibers, especially human hair, from damage by atmospheric action.

With reference to preferred inventive uses, the statement made concerning the preferred inventive compositions is correspondingly valid.

The following examples are intended to illustrate the subject of the invention in more detail, without limiting it.

### EXAMPLES

Care Shampoo.

| | 1 | 2 |
|---|---|---|
| Texapon K 14 S* | 50.0 | 50.0 |
| Dehyton K** | 10.0 | 10.0 |
| Plantacare 818 UP*** | 4.0 | 4.0 |
| D-Panthenol | 0.5 | 0.5 |
| Nicotinamide | | 0.3 |
| Keratec Pep^{#} | | 0.5 |
| Keratec IFP^{##} | 1.6 | 1.6 |
| Arlypon F**** | 1.2 | 1.2 |
| Sodium chloride | 1.3 | 1.3 |
| Preservative | q.s. | q.s. |
| Water | ad 100 | ad 100 |

*Lauryl myristyl ethersulfate, sodium salt (ca. 68% to 73% active substance content; INCI-name: Sodium Myreth Sulfate) (Cognis)
**Cocoamidopropyl betaine (Cognis)
***Alkyl polyglucoside (Cognis); INCI: COCO GLUCOSIDE
****Fatty alcohol (C12-C14) + 2 EO (Cognis)
^{#}Intermediary filament protein from wool, MW = 3 to 4 kDa (Croda)
^{##}Mixture of intermediary filament protein from wool, MW = 40 to 60 kDa and intermediary filament protein from wool, MW = 3 to 4 kDa (Croda)

Care Rinse.

| | 3 | 4 |
|---|---|---|
| Dehyquart F75* | 5.0 | 5.0 |
| Dimethicone 200/50 | 1 | 1 |
| Crodamol ML** | 2.0 | 2.0 |
| Isopropyl myristate | 1.0 | 1.0 |
| Paraffin oil perliquidum 15 cSt DAB 9 | 0.5 | 0.5 |
| Dehyquart A CA*** | 5.0 | 5.0 |
| Cetearyl alcohol | 3 | 3 |
| Salcare SC 96**** | 1.2 | 1.2 |
| D-Panthenol | 0.5 | 0.5 |
| Nicotinamide | | 0.3 |
| Keratec Pep^{#} | | 1.0 |
| Keratec IFP^{##} | 1.5 | 1.5 |
| Preservative | q.s. | q.s. |
| Water | ad 100 | ad 100 |

*Fatty alcohol methyltriethanolammonium methylsulfate dialkyl ester mixture (INCI name: Distearoylethyl Hydroxyethylmonium Methosulfate, Cetearyl Alcohol) (COGNIS)
**Myristyl laurate
***Cetyl-trimethylammonium chloride
****N,N,N-Trimethyl-2[(methyl-1-oxo-2-propenyl)oxy]-ethanaminium chloride homopolymer (50% Active substance; INCI-name: Polyquaternium-37 (and) Propylene glycol Dicaprilate Dicaprate (and) PPG-1 Trideceth-6) (ALLIED COLLOIDS)
^{#}Intermediary filament protein from wool, MW = 3 to 4 kDa (Croda)
^{##}Mixture of intermediary filament protein from wool, MW = 40 to 60 kDa and intermediary filament protein from wool, MW = 3 to 4 kDa (Croda)

Leave-On Hair Cure.

| | 1 | 2 | 3 | 4 |
|---|---|---|---|---|
| Dow Corning 949* | 7 | 7 | 7 | 7 |
| Ethanol denat. | 10 | 10 | 10 | 10 |
| Glycerine | 0.5 | 0.5 | 0.5 | 0.5 |
| Carbopol 980** | 0.5 | 0.5 | 0.5 | 0.5 |
| Sodium hydroxide (w = 33%) | q.s. | q.s. | q.s. | q.s. |
| Promois WK*** | 1.0 | | 1.0 | 1.0 |
| Keratec Pep^{#} | 0.3 | 0.3 | | |
| Keratec IFP^{##} | 0.5 | 1.0 | 1.0 | 1.0 |
| Pantolactone | | | 0.5 | 0.5 |
| Nicotinamide | | | | 0.3 |
| Gluadin WQ**** | | | 1.5 | |
| Preservative | q.s. | q.s. | q.s. | q.s. |
| Water | ad 100 | ad 100 | ad 100 | ad 100 |

*cationic silicone emulsion (Dow Corning)
**INCI: Carbomer (Noveon)
***INCI: Hydrolyzed Kreatin (Adinop)
****cationic protein derivative (Cognis)
^{#}Intermediary filament protein from wool, MW = 3 to 4 kDa (Croda)
^{##}Mixture of intermediary filament protein from wool, MW = 40 to 60 kDa and intermediary filament protein from wool, MW = 3 to 4 kDa (Croda)

1. A hair treating agent comprising from 0.05 to 5 wt. % of at least one corneocyte protein or corneocyte polypeptide or a structurally related protein having a molecular weight of 10 to 40 kDa.

2. The hair treating agent claim 1 wherein the corneocyte protein or the corneocyte polypeptide or a structurally related protein has a molecular weight from 12.5 to 37.5 kDa.

3. The hair treating agent claim 1 wherein the corneocyte protein or corneocyte polypeptide or a structurally related protein comprises 200 to 500 amino acids.

4. The hair treating agent claim 1 wherein the corneocyte protein(s) or corneocyte polypeptide(s) or a structurally related protein is/are selected from the group consisting of involucrin, loricrin cornifin, trichohyalin, sciellin, profilaggrin and keratolinin.

5. The hair treating agent claim 1 wherein the corneocyte protein(s) or corneocyte polypeptide(s) have at least 5% of a glycine-serine amino acid sequence.

6. The hair treating agent claim 1 wherein the corneocyte protein(s) or corneocyte polypeptide(s) have at least 5% of a glycine-serine-cysteine amino acid sequence.

7. The hair treating agent claim 1 comprising from 0.5 wt. % to 70 wt. % of anionic surfactant, a nonionic surfactant, a cationic surfactant, an amphoteric surfactant or a combination thereof based on the weight of the hair treating agent.

8. The hair-treatment composition of claim 1 further comprising from 0.1 to 5% by weight of vitamins, provitamins and vitamin precursors from the vitamin groups A, B, C, E, F and H.

9. The hair-treatment composition of claim 1 further comprising from 0.1 to 10% by weight of a silicone.

10. The hair-treatment composition of claim 1 wherein the silicone of component b) is a compound of the formula I
(CH₃)₃Si—[O—Si(CH₃)₂]_{X}—O—Si(CH₃)₃ (1)
wherein x is a number from 0 to 100.

11. The hair-treatment composition of claim 1 wherein the silicone of component b) is an amino-functional silicone of the formula (II)
R′ₐG₃₋ₐ—Si(OSiG₂)ₙ—(OSiG_{b}R′_{2-b)m}—O—SiG₃₋ₐ—R′ₐ (II),
wherein:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number between 0 and 3;
b is a number between 0 and 1;
m and n are numbers whose sum (m+n) is between 1 and 2000;
R′ is a monovalent radical selected from
-Q-N(R″)—CH₂—CH₂—N(R″)₂
-Q-N(R″)₂
-Q-N⁺(R″)₃A⁻
-Q-N⁺H(R″)₂ A⁻
-Q-N⁺H₂(R″)A⁻
-Q-N(R″)—CH₂—CH₂—N⁺R″H₂A⁻,
where each Q is a chemical bond, —CH₂—, —CH₂—CH₂—, —CH₂CH₂CH₂—, —C(CH₃)₂—, CH₂CH₂CH₂CH₂—, —CH₂C(CH₃)₂—, —CH(CH₃)CH₂CH₂—, R″ is identical or different radicals from the group —H, -phenyl, -benzyl, —CH₂—CH(CH₃)Ph, the C₁₋₂₀-alkyl radicals, and A represents an anion selected from chloride, bromide, iodide or methosulfate.

12. The hair-treatment composition of claim 1 wherein the silicone of component b) is a compound of the formula III 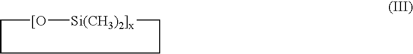
wherein x is a number from 3 to 200.

13. The hair-treatment composition of claim 1 the silicone of component b) is a compound of the formula IV
R₃Si—[O—SiR₂]ₓ—(CH₂)ₙ—[O—SiR₂]_{y}—O—SiR₃ (IV)
wherein each of R₂ and R₃ is independently —H, -phenyl, -benzyl, —CH₂—CH(CH₃)Ph, a C₁₋₂₀-alkyl radical; and each of x and y is independently a number from 0 to 200.

14. The hair-treatment composition of claim 1 further comprising a water soluble silicone.

15. A method of treating keratin fibers comprising contacting the fibers with an effective amount of a composition comprising corneocyte proteins or corneocyte polypeptides having a molecular weight of 10 to 40 kDa fiber treatment composition according to the invention which results in the improvement of at least one of the following properties
tensile strength of keratin fibers;
stabilization of the moisture balance of keratin fibers;
combability of keratin fibers;
delay in the aging process of keratin fibers;
restructurability of keratin fibers, during or after the permanent waving process;
reduction in the decrease in elasticity of keratin fibers, in the case of damage as a result of atmospheric effects.

16. A method of treating hair comprising contacting the hair with an effective amount of a composition comprising corneocyte proteins or corneocyte polypeptides having a molecular weight of 10 to 40 kDa hair treatment composition according to the invention which results in the improvement of at least one of the following properties
tensile strength of human hair;
stabilization of the moisture balance of human hair;
combability of human hair;
delay in the aging process of human hair;
restructurability of human hair, during or after the permanent waving process;
reduction in the decrease in elasticity of human hair, in the case of damage as a result of atmospheric effects.
